(19) United States
(12) Patent Application Publication
(10) Pub. No.: US 2015/0230697 A1
(43) Pub. Date: Aug. 20, 2015

(54) FLEXIBLE MASTER - SLAVE ROBOTIC ENDOSCOPY SYSTEM

(71) Applicants: Nanyang Technological University, Singapore (SG); National University of Singapore, Singapore (SG)

(72) Inventors: Soo Jay Louis Phee, Singapore (SG); Van An Huynh, Singapore (SG); Zheng Wang, Singapore (SG); Isaac David Penny, Singapore (SG); Khek Yu Ho, Singapore (SG)

(21) Appl. No.: 14/429,132

(22) PCT Filed: Sep. 19, 2013
(86) PCT No.: PCT/SG2013/000408

§ 371 Date: Mar. 18, 2015

Related U.S. Application Data
(60) Provisional application No. 61/703,241, filed on Sep. 19, 2012.

(51) Int. Cl. A61B 1/012; A61B 1/005; A61B 1/05; A61B 1/018

(52) U.S. Cl. CPC A61B 1/0125; A61B 1/018; A61B 1/0051; A61B 1/05; Y10S 901/41; Y10S 901/02

(57) ABSTRACT

A master-slave robotic endoscopy system includes a flexible primary endoscope probe having at least one tool channel for carrying a tendon-sheath driven robot arm and corresponding end effector, and a secondary endoscope probe channel for carrying an imaging endoscope. The imaging endoscope provides enhanced image capture range relative to a distal end of the primary endoscope probe by way of a secondary endoscope probe channel distal opening proximally offset from the primary endoscope probe distal end; a ramp structure distally carried by the primary endoscope probe; and/or one or more actuatable distal imaging endoscope regions. Robot arms can include joint primitives that enable robot arm/end effector manipulation in accordance with intended degrees of freedom. A set of quick connect/disconnect interfaces couple an actuation controller to one or more actuation assemblies insertable into the tool channel(s), where each actuation assembly includes tendon-sheath elements, a robot arm, and its corresponding end effector.

### TECHNICAL FIELD

The present disclosure relates to master-slave robotic endoscopy systems in which (a) a flexible primary endoscope probe carries a secondary endoscope probe configured for enhanced positioning relative to a distal end of the primary endoscope probe; (b) tendon-sheath driven robot arms carrying end effectors include one or more types of joint primitives that enable robot arm/end effector manipulation in accordance with intended degrees of freedom; and/or (c) a quick connect/disconnect interface couples an actuation controller an actuation assembly including tendon-sheath elements, a robot arm, and a corresponding end effector insertable into the primary endoscope probe.

### BACKGROUND

Surgical robotics has enabled a revolution in surgical techniques, particularly with respect to minimally invasive surgery. The advent of flexible robotic endoscopy has enabled procedures such as Natural Orifice Transluminal Endoscopic Surgery (NOTES) or “incisionless” surgical procedures that do not require a percutaneous access site into the body, whereby a flexible robotic endoscope is inserted into a natural orifice of a subject, such as the subject's mouth, and is further navigated within or along a natural internal passageway such as portions of the subject's digestive tract until a distal end of the endoscope is positioned at or proximate to a target site of interest within the subject. Once the distal end of the endoscope is positioned at the target site, a surgical intervention can be performed by way of one or more robot arms and corresponding end effectors that are carried by the endoscope, and which are translatable and manipulable beyond the endoscope's distal end. A representative example of a master-slave flexible robotic endoscope system is described in International Patent Application

### DISCLOSURE/DESCRIPTION

### Technical Problems

It is desirable to include or incorporate an imaging device such as an imaging endoscope in a flexible robotic endoscopy system, such that images can be captured and provided to a surgeon as real-time visual feedback while the surgeon performs a surgical procedure by way of one or more robot arms and end effectors corresponding thereto. Unfortunately, the manner in which imaging devices are incorporated into existing robotic endoscopy systems does not readily facilitate the capture of images at or very near the distal end of the endoscope, and/or within or across a sufficient spatial image capture range in an environment in which the distal end of the endoscope is disposed. Existing flexible robotic endoscopy systems do not provide a sufficiently or highly compact endoscope apparatus having an overall straightforward or conceptually simple and mechanically robust structure, which provides an imaging device having a suitably positioned or appropriately controllable field of view that enhances or maximizes image capture range.

Additionally, existing flexible robotic endoscopy systems fail to provide adequate or sufficiently selectable manners in which an endoscope and an imaging device carried thereby can be controlled by an individual other than the surgeon or clinician who is performing a surgical procedure by way of directing the control of the robot arm(s) and end effector(s).

It can further be desirable to provide a flexible endoscopic instrument with shape-locking capabilities. However, existing shape-lockable flexible endoscopy systems tend to be needlessly complex, and/or fail to provide a manner by which shape-locking can be selectively controlled by an individual other than the surgeon or clinician.

In addition to or beyond the foregoing, robot arms in current flexible robotic endoscopy systems tend to be unnecessarily structurally complex (and thus can have unnecessarily high parts count and greater cost), and may not be easily designed to provide intended or desired types of motion through a large number of Degrees of Freedom (DOF).

Finally, it is also desirable to provide a manner by which a flexible robotic endoscopy system can be removably, reliably, and rapidly coupled to and decoupled from an actuation system that drives the robot arms and end effectors. Existing flexible robotic endoscopy systems lack a suitable interface by which such coupling/decoupling can occur.

### Technical Solutions

This invention according to claim 1 is an endoscopy apparatus having a primary endoscope probe comprising an elongate flexible body having a length, a central axis, a proximal end, a distal end, and plurality of channels therewithin extending away from the proximal end and toward the distal end, the plurality of channels including: (a) at least one tool channel configured for receiving an endoscopy tool, each tool channel having a proximal opening and a distal opening; and (b) a secondary endoscope probe channel configured for carrying a secondary endoscope probe, the secondary endoscope probe channel having a central axis, a proximal opening, and a distal opening, wherein the distal opening of the secondary endoscope probe channel is proximally offset away from the distal end of the primary endoscope probe.

This invention according to claim 2 has, in the endoscopy apparatus of claim 1, a characteristic in that the distal opening of the secondary probe channel is proximally offset away from the distal end of the primary endoscope probe by up to 15% of the length of the primary endoscope probe.

This invention according to claim 3 has, in the endoscopy apparatus of claim 1, a characteristic in that the distal opening of the secondary probe channel is proximally offset away from the distal end of the primary endoscope probe by up to 10% of the length of the primary endoscope probe.

This invention according to claim 4 has, in the endoscopy apparatus of claim 1, a characteristic in that the endoscopy apparatus further includes: (a) an actuation assembly disposed within a tool channel of the at least one tool channel, the actuation assembly including an end effector and a set of actuation elements configured for controlling the end effector, the actuation assembly translatable along the primary endoscope probe central axis such that the end effector is disposable within a target environment beyond the distal end of the primary endoscope probe; and (b) a secondary endoscope probe carried within the secondary endoscope probe channel, the secondary endoscope probe having a distal end displaceable beyond the distal opening of the secondary endoscope probe channel, wherein the secondary endoscope probe comprises an imaging endoscope configured for capturing images of the end effector within the target environment beyond the distal end of the primary endoscope probe, and wherein the imaging endoscope includes at least one of: at least one controllable region configured for enabling controllable displacement of the imaging endoscope toward or away from the primary endoscope probe central axis, and an image capture module having a field of view disposed toward the central axis of the primary endoscope probe.

This invention according to claim 5 has, in the endoscopy apparatus of claim 4, a characteristic in that the imaging endoscope is configured for capturing anterograde and retrograde views of end effector operation within the target environment.

This invention according to claim 6 has, in the endoscopy apparatus of claim 4, a characteristic in that the at least one controllable region is configured for enabling heave displacement of the imaging endoscope relative to the primary endoscope probe central axis.

This invention according to claim 7 has, in the endoscopy apparatus of claim 6, a characteristic in that the at least one controllable region is further configured for enabling sway displacement of the imaging endoscope relative to the primary endoscope probe central axis.

This invention according to claim 8 has, in the endoscopy apparatus of claim 4, a characteristic in that the imaging endoscope includes a plurality of distinct controllable regions.

This invention according to claim 9 has, in the endoscopy apparatus of claim 9, a characteristic in that the imaging endoscope includes or is an S-bend endoscope.

This invention according to claim 10 has, in the endoscopy apparatus of claim 4, a characteristic in that the imaging endoscope is rotatable about a central or longitudinal axis thereof.

This invention according to claim 11 has, in the endoscopy apparatus of claim 4, a characteristic in that the endoscopy apparatus of claim further includes a ramp structure positioned proximate to the distal end of the primary endoscope probe, where the ramp structure is configured for receiving the imaging endoscope and guiding a central axis of the imaging endoscope toward or away from the central axis of the primary endoscope probe to thereby facilitate heave displacement of the imaging endoscope relative to the central axis of the primary endoscope probe.

This invention according to claim 12 has, in the endoscopy apparatus of claim 11, a characteristic in the ramp structure is controllably displaceable in a direction parallel to the central axis of the primary endoscope probe.

This invention according to claim 13 has, in the endoscopy apparatus of claim 4, a characteristic in that the image capture module field of view is disposed toward the central axis of the primary endoscope probe by way of one of: (a) a beveled face carrying a lens element and positioned at a non-normal angle relative to the central axis of the secondary endoscope probe; and (b) a rotatable housing carrying the lens element, the rotatable housing controllably displaceable about an axis of rotation transverse to the central axis of the primary endoscope probe.

This invention according to claim 14 has, in the endoscopy apparatus of claim 13, a characteristic in that the distal end of the primary endoscope probe is configured for mating engagement with the rotatable housing, wherein the rotatable housing is displaceable beyond the distal end of the primary endoscope probe.

This invention according to claim 15 is an endoscopy apparatus including: (a) a primary endoscope probe having an elongate flexible body having a central axis, a proximal end, a distal end, and plurality of channels therewithin extending away from the proximal end and toward the distal end of the primary endoscope probe, the plurality of channels including: (i) at least one tool channel having a proximal opening and a distal opening; and (ii) a secondary endoscope probe channel configured for carrying a secondary endoscope probe, the secondary endoscope probe channel having a central axis, a proximal opening, and a distal opening; and (b) a ramp structure positioned proximate to the distal end of the primary endoscope probe and configured for receiving the secondary endoscope probe and guiding the central axis of the central axis of the secondary endoscope probe toward or away from the central axis of the primary endoscope probe to thereby facilitate heave displacement of the secondary endoscope probe relative to the central axis of the primary endoscope probe.

This invention according to claim 16 has, in the endoscopy apparatus of claim 15, a characteristic in that the ramp structure is controllably displaceable in a direction parallel to the central axis of the primary endoscope probe.

This invention according to claim 17 is an imaging endoscope including a flexible body having a length, a central axis along its length, a proximal end, and a distal end; and an image capture module disposed at the distal end of the flexible body and having a field of view that is controllably positionable toward and away from the central axis of the flexible body by way of a rotatable housing having an axis of rotation transverse to the central axis of the flexible body.

This invention according to claim 18 is an endoscopy apparatus including a primary endoscope probe comprising an elongate flexible body having an exterior shape, a central axis, a proximal end, a distal end, and at least one tool channel therein extending away from the proximal end toward the distal end of the body, each tool channel having a proximal opening and a distal opening, wherein a distal portion of the primary endoscope probe is segmented into: (a) a tool channel member comprising a distal extension of a first cross sectional portion of the body, the tool channel member having a distal end carrying the distal opening of each tool channel of the at least one tool channel; and (b) a secondary probe member comprising a distal extension of a second cross sectional portion of the body, the secondary probe member having a distal end carrying an image capture module, the secondary probe member configured for selectable (i) position locking adjacent to the tool channel member, and (ii) positioning of the image capture module away from the tool channel member by way of heave displacement of the image capture module away from the central axis of the body, wherein the distal end of the tool channel member and the distal end of the secondary probe member terminate at the distal end of the body when the secondary probe member is position locked adjacent to the tool channel member.

This invention according to claim 19 has, in the endoscopy apparatus of claim 18, a characteristic in that the secondary probe member includes a proximal controllable region configured for enabling heave displacement of the image capture module away from the central axis of the body.

This invention according to claim 20 has, in the endoscopy apparatus of claim 19, a characteristic in the secondary probe member includes a distal controllable region configured for selectively orienting a field of view of the image capture module toward the central axis of the body.

This invention according to claim 21 has, in the endoscopy apparatus of claim 18, a characteristic in that the tool channel member and the secondary probe member each have an outer surface that uniformly maintain the exterior shape of the body from the proximal end of the body to the distal end of the body.

This invention according to claim 22 has, in the endoscopy apparatus of claim 4, a characteristic in that positioning of the primary endoscope probe and positioning of the secondary endoscope probe is controllable by an interface coupled to the proximal end of the primary endoscope probe, and wherein positioning of the robot arm is controllable by a master controller or console disposed remote from the primary endoscope probe and the interface coupled to the proximal end of the primary endoscope probe.

This invention according to claim 23 has, in the endoscopy apparatus of claim 22, a characteristic in that positioning of the secondary endoscope probe is further selectably controllable by the master controller.

This invention according to claim 24 is a selectively shape lockable endoscopy apparatus including: (a) a primary endoscope probe having an elongate flexible body having a length, a central axis, a proximal end, a distal end, and at least one tool channel therein extending away from the proximal end toward the distal end of the body, each tool channel having a proximal opening and a distal opening; (b) a plurality of tensionable cables carried internal to the flexible body and configured for selectively shape locking at least one shape lockable section of the flexible body in response to applied tension during navigation of the flexible body toward and into a target environment, wherein the plurality of cables is coupled to at least one of (i) a plurality of actuated joints disposed at each predetermined shape lockable section, and (ii) the elongate flexible body at predetermined longitudinal distances along the flexible body length to effectuate shape locking in response to applied tension; (c) an actuation assembly disposed within a tool channel of the at least one tool channel, the actuation assembly including a robot arm carrying an end effector and a set of actuation elements configured for controlling the robot arm and end effector; (d) an interface coupled to the proximal end of the flexible body and configured for controlling navigation of the flexible body; and a master controller disposed remote from the flexible body and the interface coupled to the interface coupled to the proximal end of the flexible body, and configured for controlling the operation of the robot arm and the end effector.

This invention according to claim 25 has, in the endoscopy apparatus of claim 24, a characteristic the plurality of tensionable cables is coupled to each of a plurality of actuated joints disposed at each predetermined shape lockable section, and the elongate flexible body at predetermined longitudinal distances along the flexible body length.

This invention according to claim 26 is a robot arm assembly including an end effector, the robot arm assembly configured for selectively positioning the end effector in accordance with at least one degree of freedom (DOF), the robot arm assembly having a central axis and including a plurality of joint primitives, each joint primitive disposed at a predetermined position along a length of the robot arm assembly, each joint primitive configured for selectively enabling motion corresponding to a particular DOF, each joint primitive actuatable by way of a set of tendons, the plurality of joint primitives including at least two of: (a) a vertebra joint primitive configured for displacing a first segment of the robot arm assembly toward or away from the central axis of the robot arm assembly relative to a second segment of the robot arm assembly, the vertebra joint primitive including: (i) a proximal body portion corresponding to the first segment of the robot arm assembly, the proximal body portion having a cross sectional area and a central axis; and (ii) a distal body portion corresponding to the second segment of the robot arm assembly, the distal body portion carried by the proximal body portion by way of pivotable mating engagement relative to the proximal body portion, the distal body portion having a cross sectional area and a central axis alignable with the central axis of the proximal body portion, the distal body portion comprising a first tendon coupling portion couplable to a first tendon, and a second tendon coupling portion couplable to a second tendon, wherein the central axis of the distal body portion is selectively alignable with the central axis of the proximal body portion and the central axis of the robot arm assembly by way of application of forces to the first and second tendons; (b) a rotational joint primitive configured for rotating a third segment of the robot arm assembly in a clockwise or counterclockwise relative to the central axis of the robot arm assembly, the rotational joint primitive including: (i) a drum member having an outer periphery, a cross sectional area, and an axis of rotation perpendicular to the cross sectional area; and (ii) a third tendon wrapped around the outer periphery of the drum member and configured to rotate the drum member in response to a pulling force differentially applied to a first end of the third tendon relative to a second end of the third tendon; and (c) a revolute joint primitive configured for pivoting a fourth segment of the robot arm assembly relative to a fifth segment of the robot arm assembly, the revolute joint primitive comprising a body revolvable in a first direction relative to the central axis of the robot arm assembly by way of a first pulling force applied to a fourth tendon secured to the body and revolvable in a second direction opposite to the first direction by way of a second pulling force applied to a fifth tendon secured to the body.

This invention according to claim 27 has, in the endoscopy apparatus of claim 26, a characteristic in that the robot arm assembly is movable in a plurality of DOF corresponding to at least one of shoulder medial rotation, elbow flexion/extension, forearm supination/pronation, wrist flexion/extension, and finger opposition/ reopposition. This invention according to. claim 28 has, in the endoscopy apparatus of claim 26, a characteristic in the robot arm assembly is configured for motion in eight DOF.

This invention according to claim 29 is an endoscopy apparatus including a quick release assembly configured for: (a) receiving (i) a first set of flexible tendon-sheath elements corresponding to an actuation controller configured for linearly driving tendons within the first set of tendon-sheath elements, and (ii) a second set of flexible tendon-sheath elements corresponding to an actuation assembly insertable into an endoscope probe and including the second set of tendon-sheath elements and a robot arm carrying an end effector controllable by way of linear motion of tendons within the second set of tendon-sheath elements; and (b) converting linear motion of tendons within the first set of tendon-sheath elements into rotational motion, and converting the rotational motion into linear motion of tendons within the second set of tendon-sheath elements to facilitate control of the robot arm and the end effector in response to linear motion of tendons within the first set of tendon-sheath elements.

This invention according to claim 30 has, in the endoscopy apparatus of claim 29, a characteristic in that the quick release assembly carries a portion of a surgical drape that facilitates environmental isolation between (a) the actuation controller and the first set of tendon-sheath elements, and (b) the actuation assembly and the endoscope probe.

This invention according to claim 31 has, in the endoscopy apparatus of claim 29, a characteristic in that the quick release assembly includes: an actuator-side interface configured to receive the first set of flexible tendon-sheath elements; and an endoscope-side interface configured to receive the second set of tendon-sheath elements, wherein the actuator-side interface and the endoscope-side interface are configured for detachable mechanical coupling with each other.

This invention according to claim 32 has, in the endoscopy apparatus of claim 31, a characteristic in that the quick release assembly further includes an intermediate interface configured for detachable mating engagement with each of the actuator-side interface and the endoscope-side interface, wherein the conversion of linear motion of tendons within the first set of tendon-sheath elements into rotational motion and the conversion of the rotational motion into linear motion of tendons within the second set of tendon-sheath elements occurs by way of the intermediate interface.

This invention according to claim 33 has, in the endoscopy apparatus of claim 32, a characteristic in that the intermediate interface is configured for snap-fit engagement with each of the actuator-side interface and the endoscope-side interface.

This invention according to claim 34 has, in the endoscopy apparatus of claim 32, a characteristic in that the intermediate interface carries a portion of a surgical drape that facilitates environmental isolation between (a) the actuation controller and the first set of tendon-sheath elements, and (b) the actuation assembly and the endoscope probe.

This invention according to claim 35 has, in the endoscopy apparatus of claim 29, a characteristic in that the quick release assembly carries a set of sensors configured to detect tendon forces and/or tendon elongation.

### Advantageous Effects

According to the invention disclosed in claim 1, the secondary endoscope probe channel is proximally offset or set back away from the distal end of the primary endoscope probe. As a result, a secondary endoscope probe carried within the secondary endoscope probe channel (e.g., internal to a main body or overall outer body profile of the primary endoscope probe) and which is configured for heave displacement relative to the primary endoscope probe can be displaced further away from the central axis primary endoscope probe in the vicinity of the distal end of the primary endoscope probe in association with or following (a) a small or relatively small amount of surge displacement of the secondary endoscope probe beyond the secondary endoscope probe channel's distal opening, toward, to, and/or past the distal end of the primary endoscope probe, and (b) heave displacement of the secondary endoscope probe away from the central axis of the primary endoscope probe. Consequently, when the secondary endoscope probe includes or is an imaging endoscope, the imaging endoscope can capture images of an environment at or very near to which the distal end of the primary endoscope probe resides. The captured images can provide accurate visual information with regard to the positioning of the distal end of the primary endoscope probe relative to its external environment, and/or the positioning and operation of portions of one or more actuation assemblies (e.g., including a set of robot arms and end effectors) at or very near the distal end of the primary endoscope probe. Such visual information was previously not readily obtainable by way of a single endoscopy apparatus, and in particular, an endoscopy apparatus having a conceptually simple and mechanically robust overall structure.

According to the invention disclosed in claim 2, the secondary endoscope probe channel is proximally offset or set back away from the distal end of the primary endoscope probe by up to 15% of the length of the primary endoscope probe's length, and according to the invention disclosed in claim 3, the proximal offset is up to 10% of the primary endoscope probe's length. This proximal offset distance can be predetermined or selected in accordance with endoscopy apparatus shape/dimensions, the type of actuation assemblies (e.g., the type(s) of robot arms and/or end effectors) under consideration, and/or the nature of an endoscopic intervention under consideration. When the secondary endoscope probe includes an imaging device, this proximal offset distance can facilitate accurate endoscopic imaging (a) at or very near and beyond the distal end of the primary endoscope probe, and additionally (b) accurate imaging at least slightly proximal to the distal end of the primary endoscope probe when the primary endoscope probe is positioned at an intended destination at which a surgical intervention is occurring and the robot arm and end effector are performing a procedure. An imaging device carried by a secondary endoscope probe disposed within the secondary endoscope probe channel can thus capture images that provide visual information about the state of an environment in which the endoscopy tool is performing a procedure beyond the distal end of the primary endoscope probe, and/or the state of an environment at, very near, and/or at least slightly proximal to the distal end of the primary endoscope probe for purpose of monitoring the progress of the procedure and the condition/state of such environments during the procedure without requiring primary endoscope probe repositioning, while only slightly or minimally disturbing the environment at and around the distal end of the primary endoscope probe.

According to the invention disclosed in claim 4, the secondary endoscope probe includes an imaging endoscope having at least one of (a) one or more controllable regions configured for enabling heave displacement of the imaging endoscope toward/away from the primary endoscope probe central axis, and (b) an image capture module having a field of view disposed toward the central axis of the primary endoscope probe. The controllable regions and/or the image capture module can facilitate the selective positioning or biasing of the imaging endoscope's field of view toward the primary endoscope probe's central axis, and selectively proximally/distally within portions of the primary endoscope probe's external environment. As a result, the imaging endoscope can more readily capture images within an entire target environment of interest, at or very near and beyond the distal end of the primary endoscope probe.

In related manners, according to the invention disclosed in claim 5, the imaging endoscope is configured for capturing anterograde and retrograde views of operations performed by an end effector operating within a target environment beyond the distal end of the primary endoscope probe. According to the invention disclosed in claims 6 and 7, the controllable region(s) enable selective heave displacement and possibly sway displacement of the imaging endoscope relative to the primary endoscope probe central axis; and according to the invention disclosed in claim 8 the imaging endoscope includes a plurality of distinct controllable regions, such as in an S-bend type of endoscope according to the invention disclosed in claim 9. Such types of controllable region configurations enable increased control over the positioning of the imaging endoscope to thereby facilitate greater positional adjustability and enhanced image capture range.

According to the invention disclosed in claim 10, the imaging endoscope is configured for controllable rotation about its central/longitudinal axis. Such rotation provides the imaging endoscope with an additional type of maneuverability for capturing images within a spatial volume corresponding to a target environment in which the distal end of the primary endoscope probe is disposed, and in which one or more robot arms and corresponding end effectors can operate.

According to the invention disclosed in claim 11, the ramp structure at or near the primary endoscope's distal end can receive the imaging endoscope, and guide the imaging endoscope toward/away from the primary endoscope probe's central axis (as the imaging endoscope is surged toward/away from the distal end of the primary endoscope probe) to thereby facilitate heave displacement of the imaging endoscope with respect to the primary endoscope probe's central axis. The ramp structure thus enhances an extent to which the imaging endoscope can be displaced away from the primary endoscope probe's central axis, thus facilitating an increased imaging range for the imaging endoscope. According to the invention disclosed in claim 12, the ramp structure is movable parallel to or along the primary endoscope probe's central axis. Such ramp movability enables further adjustability in the extent to which the imaging endoscope can be heave displaced away from the primary endoscope probe's central axis.

According to the invention disclosed in claim 13, the image capture module field of view is oriented toward the primary endoscope probe's central axis by a beveled face or a rotatable housing carrying a lens element. The beveled face predisposes the lens element toward the primary endoscope probe's central axis; and the rotatable housing enables selective orientation of the lens element toward this central axis. In each case, the ability of the imaging endoscope to capture images of end effector positioning and operation within the primary endoscope probe's external environment is enhanced. According to the invention disclosed in claim 14, the rotatable housing and the distal end of the primary endoscope probe are configured for mating engagement with each other, resulting in a compact and space efficient endoscopy apparatus. Furthermore, the rotatable housing is displaceable beyond the distal end of the primary endoscope probe, thus further enhancing the spatial range within and across which the imaging endoscope can capture images.

According to the invention disclosed in claim 15, the ramp structure proximate to the distal end of the primary endoscope probe enhances an extent to which the secondary endoscope probe can be displaced away from the primary endoscope probe's central axis, thus facilitating an increased spatial positioning range for the secondary endoscope probe. According to the invention disclosed in claim 16, the ramp structure is controllably movable parallel to this central axis, which further enhances the extent to which the positioning of the secondary endoscope probe relative to the primary endoscope probe can be adjusted.

According to the invention of claim 17, the field of view of the imaging endoscope's rotatable camera can be controllably or selectively positioned toward/away from the imaging endoscope's central axis. Such rotatable positioning of the field of view significantly enhances the image capture range of the imaging endoscope with respect to an external environment in which the image capture module is disposed, without requiring (although not preventing) the imaging endoscope to be configured for heave and/or sway displacement. Consequently, such an imaging endoscope can realize an enhanced imaging range without requiring an enhanced displacement range relative to its central axis.

According to the invention of claim 18, the primary endoscope probe's distal end is segregated into a tool channel member and a secondary probe member carrying an image capture module, and which can be selectively position locked relative to or against the tool channel member or displaced away from the tool channel member. As a result, the image capture module can be heave displaced above the tool channel member, such that the image capture module can more effectively capture images of end effector positioning and operation in a target environment beyond the distal end of the primary endoscope probe.

According to the invention of claims 19 and 20, the secondary probe member includes a proximal controllable region, and a distal controllable region, respectively. Such controllable regions facilitate enhanced selective positioning of the image capture module relative to the central axis of the primary endoscope probe, and thus facilitate a greater image capture range for the image capture module.

According to the invention of claim 21, when the secondary probe member is position locked adjacent to (e.g., against) the tool channel member, outer surfaces of the secondary probe member and the tool channel member uniformly maintain the shape of the primary endoscope probe body between its proximal end and distal end. As a result, when position locked, the secondary probe member does not interfere with primary endoscope probe insertion into or navigation through an intended environment.

According to the invention of claim 22, positioning/navigation of the primary and secondary endoscope probes are positionable by way of an interface (e.g., an endoscopist interface) coupled to the proximal end of the primary endoscope probe; and positioning of the robot arm is controllable by way of the remote master controller (e.g, a surgeon interface). As a result, an endoscopist present in the operating theater with a subject/patient can focus on or be responsible for navigation of the primary endoscope probe, and a surgeon remote from the subject/patient can focus on or be responsible for carrying out an intended procedure by way of robot arm(s) and end effector(s) carried by the primary endoscope probe. According to the invention of claim 23, the positioning of the secondary endoscope probe is selectably controllable by the master controller. Consequently, the surgeon can specifically position the secondary endoscope probe themselves when desired or needed.

According to the invention of claim 24, the primary endoscope probe's body is tensionable by way of cables coupled to at least one of (a) actuated joints disposed at each predetermined shape lockable section, and (b) the elongate flexible body at predetermined longitudinal distances along the flexible body length to effectuate shape locking in response to applied tension. Navigation of the primary endoscope probe body is controllable by way of an interface (e.g., an endoscopist interface) coupled to its proximal end. A robot arm and an end effector coupled thereto, which are carried by the primary endoscope probe body, are controllable by way of a master controller (e.g., a surgeon interface) disposed remote from the primary endoscope probe body and the interface coupled to the proximal end thereof. As a result, an endoscopist present in the operating theater with a subject/patient can focus on or be responsible for navigation of the primary endoscope probe, and a surgeon remote from the subject/patient can focus on or be responsible for carrying out an intended procedure by way of robot arm(s) and end effector(s) carried by the primary endoscope probe. The endoscopist can thus additionally be responsible for selectively tensioning the cables to shape lock the primary endoscope probe body (e.g., by way of the endoscopist interface) once the distal end of the primary endoscope probe body has reached a target destination or environment. According to the invention of claim 25, the cables are coupled to each of the actuated joints and the primary endoscope probe body.

According to the invention of claim 26, the robot arm assembly includes multiple distinct types of joint primitives that provide foundational joint elements that can be incorporated into robot arms, including two of more of vertebra joint primitives, rotational joint primitives, and revolute joint primitives. Such joint primitives enable the construction of a structurally straightforward and hence reduced parts count/lower cost robot arm assembly that is manipulable in accordance with a predetermined, intended, or desired number of degrees of freedom (DOFs). According to the invention of claim 27, the robot arm assembly is movable in DOF corresponding to two or more of shoulder medial rotation, elbow flexion/extension, forearm supination/pronation, wrist flexion/extension, and finger opposition/reopposition; and according to the invention of claim 28, the robot arm assembly is configured for motion in eight DOF. Thus, the construction of a robot arm assembly by way of such joint primitives can result in a highly positionable/manipulable robot arm assembly.

According to the invention of claim 29, the quick release assembly includes a plurality of selectively engageable/releaseable elements configured for converting linear tendon motion corresponding to a first set of flexible tendon-sheath elements (e.g., received from an actuation controller) into rotational motion, and is further configured for converting this rotational motion into linear tendon motion corresponding to a second set of flexible tendon-sheath elements (e.g., which form portions of the actuation assembly that enables controllable positioning/motion of a robot arm and an end effector coupled thereto). Mating engagement of the quick release assembly's engageable/releasable elements thus enables flexible tendon-sheath elements corresponding to an actuation assembly to be selectively and releaseably mechanically coupled to flexible tendon-sheath elements corresponding to an actuation assembly such that the actuation assembly can be driven by the actuation controller.

According to the invention of claim 30, the quick release assembly carries a portion of a surgical drape (e.g., a surgical or sterile barrier). The quick release assembly and its surgical drape can thus serve as an interface between non-sterile portions of an endoscopy system, such as an actuation controller and tendon-sheath elements directly coupled thereto, and sterile portions of the endoscopy system, such as the actuation assembly and the endoscope probe.

According to the invention of claim 31, the plurality of selectively engageable/releaseable elements includes an actuator-side interface and an endoscope-side interface, thus providing a structurally simple mechanical assembly that can be detachably mated together such that actuation controller tendons can drive actuation assembly tendons.

According to the invention of claim 32, the quick release assembly also includes an intermediate interface configured for converting linear tendon motion corresponding to actuation controller tendons into rotational motion, and converting this rotational motion into linear motion that drives actuation assembly tendons. According to the invention of claim 33, the intermediate interface is configured for snap-fit engagement with the quick release assembly's actuator-side and endoscope-side interfaces; and according to claim 34, the intermediate interface carries a portion of the surgical drape. The actuator-side and endoscope-side quick release interface elements can thus be conveniently engaged with and disengaged from the intermediate interface, on non-sterile and sterile sides of the intermediate interface, respectively.

According to the invention of claim 35, the quick release assembly carries a set of sensors configured to detect tendon forces and/or elongation, remote from the end effector(s), the robot arm(s), and the primary endoscope probe; and separate or apart from an actuation controller. Such sensors can facilitate the provision of force feedback to a master console.

### BRIEF DESCRIPTION OF THE DRAWINGS

FIGS. 1A and 1B are a schematic illustration and a block diagram, respectively, of a master-slave robotic endoscopy system in accordance with an embodiment of the present disclosure.

FIG. 2 is a schematic illustration of a primary endoscope probe body configured for selective or selectable shape locking in accordance with an embodiment of the present disclosure.

FIG. 3A is a schematic illustration of a primary endoscope probe configured for carrying a secondary endoscope probe, such as an imaging and/or other type of endoscope, in accordance with an embodiment of the present disclosure.

FIGS. 3B and 3C are front views of a primary endoscope probe configured for carrying a secondary endoscope probe in accordance with embodiments of the present disclosure.

FIGS. 3D is a schematic illustration of an S-bend imaging endoscope having a first controllable region, a second controllable region, and a substantially rigid section disposed between the first and second controllable regions in accordance with an embodiment of the present disclosure.

FIG. 3E is a schematic illustration showing representative cables and representative vertebra configured for facilitating counterflex motion of the first and second controllable regions of the S-bend imaging endoscope of FIG. 3D.

FIGS. 3F-3H are schematic illustrations of a primary endoscope probe configured for carrying a bevel tip imaging endoscope having a single controllable region for articulation in accordance with an embodiment of the present disclosure.

FIG. 3I is a schematic illustration of a primary endoscope probe configured for carrying an S-bend imaging endoscope having two controllable regions in accordance with another embodiment of the present disclosure.

FIGS. 3J and 3K are schematic illustrations of a primary endoscope probe configured for carrying a bevel tip imaging endoscope having a single controllable region in accordance with other embodiments of the present disclosure.

FIG. 3L is a schematic illustration of a primary endoscope probe configured for carrying an imaging endoscope having a rotatable camera assembly in accordance with an embodiment of the present disclosure.

FIG. 3M is a schematic illustration showing particular aspects of the imaging endoscope and rotatable camera assembly of FIG. 3L.

FIGS. 4A and 4B are schematic illustrations of a primary endoscope probe that includes a secondary probe member in accordance with an embodiment of the present disclosure.

FIGS. 5A and 5B are schematic illustrations of a primary endoscope probe that includes a secondary probe member in accordance with another embodiment of the present disclosure.

FIG. 6A is a perspective view of a representative embodiment of a primary endoscope probe corresponding to FIG. 3A, which is configured for carrying a first robot arm, a second robot arm, and an S-bend imaging endoscope in accordance with an embodiment of the present disclosure.

FIG. 6B is a perspective view of a representative embodiment of a primary endoscope probe corresponding to FIG. 3F, which is configured for carrying a first robot arm, a second robot arm, and a bevel tip imaging endoscope in accordance with an embodiment of the present disclosure.

FIG. 7A is a schematic illustration of a flexible or substantially flexible disposable actuation assembly in accordance with an embodiment of the present disclosure.

FIG. 7B is a perspective schematic illustration, and FIG. 7C is a cross sectional schematic illustration of flexible or substantially flexible tendon-sheath structures within a flexible or substantially flexible disposable actuation assembly in accordance with an embodiment of the present disclosure.

FIG. 7D is a cross sectional schematic illustration of a representative relationship between the internal cross sectional area of a disposable actuation assembly 300 and the overall cross sectional area within the disposable actuation assembly 300 occupied by its tendon-sheath structures 330, which can facilitate the provision and/or maintenance of significant or substantial disposable actuation assembly flexibility.

FIG. 7E is a schematic illustration of a tendon-sheath structure that includes a sheath termination element in accordance with an embodiment of the present disclosure.

FIG. 8A is a schematic illustration of a representative vertebra joint primitive in accordance with an embodiment of the present disclosure.

FIG. 8B is a schematic illustration of a representative rotational joint primitive in accordance with an embodiment of the present disclosure.

FIGS. 8C-8E are a schematic side view, cross sectional view, and top view, respectively, of a robot arm that includes vertebra joint primitives and rotational joint primitives, and which is configured for selective motion in six DOFs, in accordance with an embodiment of the present disclosure.

FIG. 9A is a schematic illustration of representative revolute joint primitives in accordance with an embodiment of the present disclosure.

FIG. 9B is a schematic illustration of a robot arm which includes multiple revolute joint primitives, and which is configured for providing eight DOF in accordance with an embodiment of the present disclosure.

FIGS. 10A and 10B are schematic illustrations of an endoscopist interface in accordance with an embodiment of the present disclosure.

FIGS. 11A-11E are schematic illustrations showing aspects of quick release interfaces in accordance with an embodiment of the present disclosure.

FIG. 11F is a schematic illustration of a tendon tensioning mechanism in accordance with an embodiment of the present disclosure.

FIG. 11G is a schematic illustration of representative mating engagement structures corresponding to quick release interfaces in accordance with an embodiment of the present disclosure.

FIG. 11H is a schematic illustration of a rotational-to-linear motion converter in accordance with another embodiment of the present disclosure.

FIG. 11I is a schematic illustration of a gimbal plate mechanical force transfer structure in accordance with an embodiment of the present disclosure.

FIG. 12 is a schematic illustration of an actuation controller in accordance with an embodiment of the present disclosure.

### DETAILED DESCRIPTION OF EXAMPLE EMBODIMENTS

In the present disclosure, depiction of a given element or consideration or use of a particular element number in a particular FIG. or a reference thereto in corresponding descriptive material can encompass the same, an equivalent, or an analogous element or element number identified in another FIG. or descriptive material associated therewith. The use of “/” in a FIG. or associated text is understood to mean “and/or” unless otherwise indicated. The recitation of a particular numerical value or value range herein is understood to include or be a recitation of an approximate numerical value or value range, for instance, within +/−10%, or +/−5% of a recited value or value range.

As used herein, the term “set” corresponds to or is defined as a non-empty finite organization of elements that mathematically exhibits a cardinality of at least 1 (i.e., a set as defined herein can correspond to a unit, singlet, or single element set, or a multiple element set), in accordance with known mathematical definitions (for instance, in a manner corresponding to that described in An Introduction to Mathematical Reasoning: Numbers, Sets, and Functions, “Chapter 11: Properties of Finite Sets” (e.g., as indicated on p. 140), by Peter J. Eccles, Cambridge University Press (1998)). In general, an element of a set can include or be a system, an apparatus, a device, a structure, an object, a process, a physical parameter, or a value depending upon the type of set under consideration.

Embodiments in accordance with the present disclosure are directed to a robotically driven master-slave endoscopy system and associated robotic endoscopy processes or procedures involving one or more of the following:
(a) a flexible or substantially flexible endoscope guide tube or probe configured for selective/selectable stiffening or shape/position locking at or along one or more portions, positions, or segments of its length, while in some embodiments maintaining or providing substantial flexibility at or along other portions, positions, or segments of its length;
(b) a flexible or substantially flexible primary, larger, multi-purpose, or general-purpose endoscope probe configured for carrying or supporting each of (i) a secondary, adjunctive, smaller, or special purpose flexible or substantially flexible endoscope probe, probe module, or probe member, portions of which can be controlled independently of the primary endoscope probe (e.g., on a selective basis), and (ii) a set of robotic/robot arms;
(c) a number of flexible or substantially flexible disposable actuation assemblies, at least some of which (i) carry tendon-sheath actuation elements; and (ii) are configured for insertion into and through the primary endoscope probe such that an endoscopy instrument or tool (e.g., a surgical instrument corresponding to an (end) effector carried by a robot arm) can extend beyond a distal end of the primary endoscope probe and be manipulated or driven by way of such tendon- sheath actuation elements;
(d) tendon-sheath driven robot arms that can include one or more types of joints, and which can be configured for carrying or coupling to various types of end effectors (e.g., grippers, pincers, hooks, forceps, knives, electrosurgery devices, needles, etc . . . ) that facilitate particular types of surgical interventions;
(e) a quick connect/disconnect or quick release interface configured for mechanically and/or electrically releasably coupling (e.g., selectively coupling and decoupling) a disposable actuation assembly and an actuation controller; and
(f) an actuation controller configured for (i) manipulating robot arms and end effectors in response to signals generated by a surgeon interface such as a master controller or control console; (ii) sensing force signals corresponding to or correlated with the movement or positioning of one or more robot arms and/or end effectors and communicating such force signals or correlates thereof (e.g., haptic feedback signals correlated with sensed forces) to the surgeon interface; and possibly (iii) controlling or selectively controlling the operation of the secondary endoscope probe, probe module, or probe member.

Depending upon embodiment details, one or more of the foregoing, or each of the foregoing, can be combined, unified, or integrated to form portions of a master-slave robotic endoscopy system.

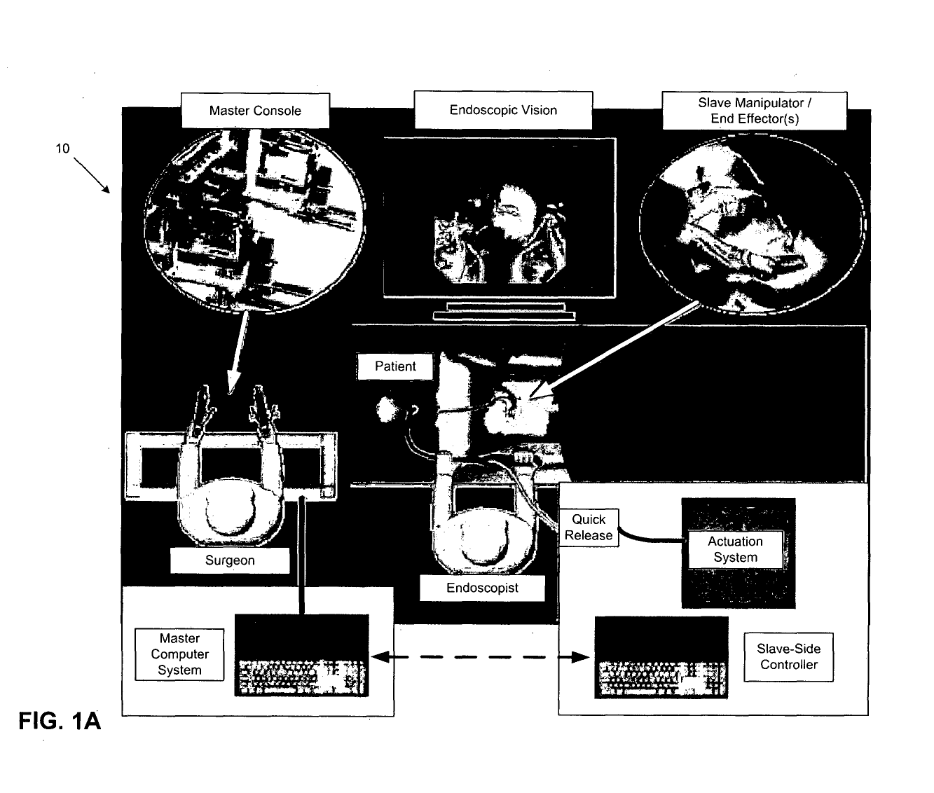

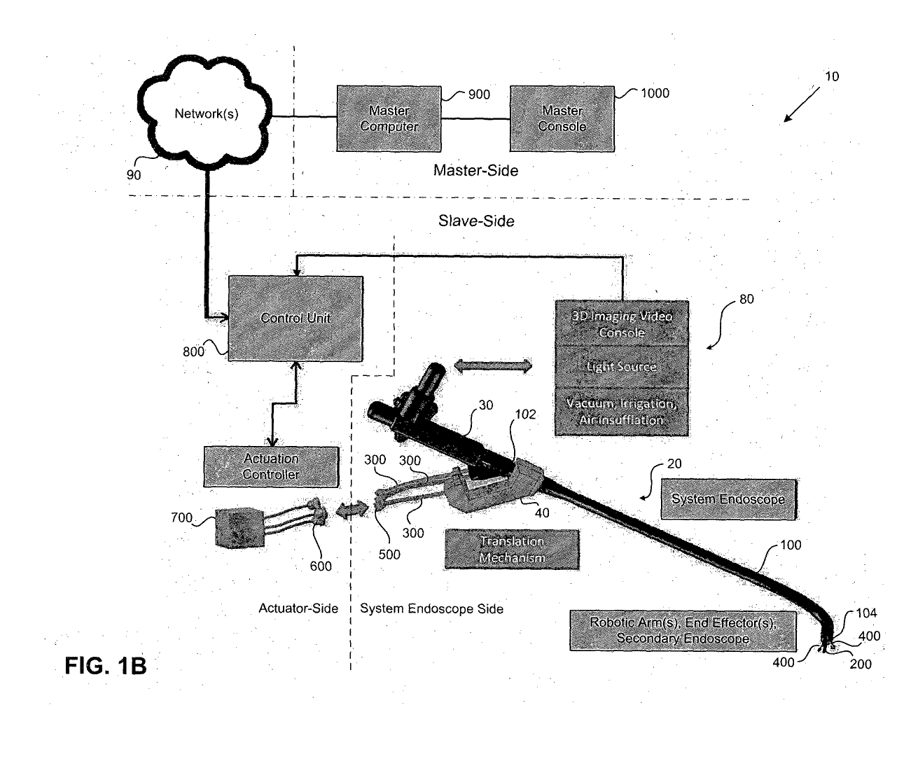

FIGS. 1A and 1B are a schematic illustration and a block diagram, respectively, of a master-slave robotic endoscopy system 10 in accordance with an embodiment of the present disclosure.

Overview of Slave-Side System Aspects

In an embodiment, a slave portion or slave-side of the system 10 includes a system endoscope 20; a support station 80; plus an actuation controller 700 and an associated slave-side control unit 800 configured for managing actuation controller operation and communicating with the master-side of the system 10, where such communication can occur by way of one or more networks 90 (e.g., a Local Area Network (LAN), a Wide Area Network (WAN), and/or the Internet).

The system endoscope 20 includes an endoscopist interface 30 and a primary endoscope probe 100. In some embodiments, the system endoscope 20 also includes a translation mechanism 40. The primary endoscope probe 100 has a proximal section or end 102 coupled/couplable to the endoscopist interface 30, such that the primary endoscope probe 100 extends away from the endoscopist interface 30 along a primary endoscope probe length to a terminal or distal section or end 104 of the primary endoscope probe 100. The primary endoscope probe 100 exhibits a cross sectional area or diameter, through which a central or longitudinal axis can be defined which extends through a center or centroid of the primary endoscope probe's cross sectional area or diameter along the primary endoscope probe length.

The endoscopist interface 30 provides a control interface that facilitates or enables endoscopist control over aspects of slave-side system operation, for instance, navigational control over the primary endoscope probe 100. As will be understood by one of ordinary skill in the relevant art, the endoscopist interface 30 includes a housing or body that provides a number of apertures, openings, or ports through which passages or channels within the primary endoscope probe 100 can be accessed. Surgical devices or instruments relevant to a surgical procedure under consideration can be inserted into and through, and withdrawn or removed from, the channels within the primary endoscope probe 100 by way of such endoscopist interface openings.

The endoscopist interface 30 also provides a common physical structure which links one or more types of adjunct endoscopy elements, devices, or subsystems to the primary endoscope probe 100. Such adjunct endoscopy elements can include a set of illumination sources (e.g., LEDs); an imaging or display console; and one or more of a suction/ vacuum, irrigation, and/or insufflation apparatus. Each adjunct endoscopy element can be associated with the support station 80. Additionally, the endoscopist interface 30 includes a number of endoscopist control elements, such as one or more buttons, knobs, switches levers, joysticks, and/or other control elements, which facilitate or enable endoscopist control over various primary endoscope probe operations, in a manner understood by one of ordinary skill in the relevant art.

The primary endoscope probe 100 is configured for carrying (i) a secondary endoscope probe, probe module, or probe member 200, as well as (ii) a set of disposable actuation assemblies 300. At least one disposable actuation assembly 300 is coupled to, supports, and/or carries a corresponding robot arm 400 that provides or is coupled to a particular type of effector or end effector suitable for performing a surgical procedure or intervention upon a subject or patient 5.

The translation mechanism 40 can be coupled to a proximal portion or the proximal end 102 of the primary endoscope probe 100, and/or a portion of the endoscopist interface 30. The translation mechanism 40 can carry portions of one or more disposable actuation assemblies 300 that reside external to yet near or generally near the endoscopist interface ports into which the disposable actuation assemblies 300 are inserted. The translation mechanism 40 is configured for selectively translating such disposable actuation assemblies 300 lengthwise or longitudinally, i.e., proximally or distally, along the central axis of the primary endoscope probe 100, relative to a maximum translation range, in response to surgeon input. More particularly, the translation mechanism 40 is configured for proximally or distally translating a disposable actuation assembly 300 along a portion of the primary endoscope probe's length, relative to the maximum translation range, to thereby respectively proximally or distally translate and position its corresponding robot arm 400 and end effector relative to the distal end 104 of the primary endoscope probe 100.

In multiple embodiments, a robot arm 400 is further drivable, manipulable, or positionable by way of tendons or tendon elements disposed within corresponding sheaths or sheath elements, where such tendon-sheath elements are carried by the disposable actuation assembly 300. A number of tendon-sheath elements associated with any given robot arm 400 is correlated with or corresponds to a number of degrees of freedom (DOF) relative to which the robot arm 400 and/or its end effector can be spatially manipulated or positioned. As will be readily understood by one of ordinary skill in the relevant art, a given robot arm's DOF indicates the collective types of translational and/or rotational motions that are supported or provided by the particular structural configuration of the robot arm 400. In a number of embodiments, the translation mechanism 40 can provide a robot arm 400 and its effector with one DOF, and a set of tendon-sheath elements coupled or linked to one or more types of joint elements or joint primitives can provide the robot arm 400 and its effector with additional DOFs, as further detailed below.

A slave-side actuation controller 700 includes a number of actuation or drive elements (e.g. motors and encoders) configured for selectively generating drive, manipulation, positioning, or displacement forces or motions (e.g., pulling forces) intended for driving or spatially manipulating/positioning/displacing the robot arm(s) 400 and end effector(s). In various embodiments, the drive forces selectively, precisely, and controllably displace or position tendon elements relative to each other in a manner that can selectively, precisely, and controllably dispose one or more portions of a tendon-sheath driven robot arm 400 and/or an end effector coupled thereto in and/or through a desired, intended, or expected spatial orientation, in a manner essentially identical, analogous, or generally analogous to that described in PCT Publication No. WO 2010/138083.

The actuation controller 700 can also include a set of force sensing units or elements configured for sensing, detecting, measuring, monitoring, and/or predicting forces applied or exerted by, and/or applied or exerted upon, portions of a robot arm 400 and/or its end effector within an environment in which the robot arm 400 is disposed. Such force sensing elements can include load sensors or load cells configured for detecting elongation and/or compressive forces communicated to a disposable actuation assembly's tendon elements by way of or in response to robot arm/end effector positioning. Aspects of the actuation controller's force sensing elements can be essentially identical, analogous, or substantially analogous to that described in PCT Publication No. WO 2010/138083.

In multiple embodiments, each disposable actuation assembly 400 is coupled/couplable to or includes a quick release structure 500 (e.g., a first or endoscope-side quick release structure) that is matingly engageable/disengageable with respect to a counterpart quick release structure 600 that is coupled/couplable to or provided by the actuation controller 700 (e.g., a second or actuator-side quick release structure). Such quick release structures 500, 600 facilitate or enable the separation, segregation or isolation of endoscope-side elements of the system 10 from actuator-side elements of the system 10, for instance, in a manner that can maintain endoscope-side system elements under pathogen controlled or sterile conditions, as further detailed below.

A quick release structure 500, 600 is configured for communicating or transferring actuation forces generated by the actuation controller 700 to the disposable actuation assembly 300, which further communicates or transfers such forces to an endoscopy instrument or tool which is disposed/disposable at and/or beyond the distal end 104 of the primary endoscope probe 100. For instance, a quick release structure 500, 600 is configured for communicating or transferring the actuation controller's robot arm drive forces to a robot arm 400, for instance, by way of communicating tendon displacement forces (e.g., pulling forces) exerted by the actuation controller 700 upon actuator-side tendon elements to corresponding endoscope-side tendon elements within a disposable actuation assembly 300. Such quick release structures 500, 600 can also be configured for communicating forces exerted upon portions of robot arms 400 and/or end effectors by tissues or objects to the actuation controller's force sensing elements, such as by way of communicating or transferring particular distortion forces to actuation-side tendon elements. Furthermore, a quick release structure 600 can provide or include an environmental isolation barrier, such as a surgical/sterile drape, which physically isolates an actuator-side environment from an endoscope-side environment (e.g., an operating theater).

The actuation controller 700 is couplable to a main control unit 800 such as a computer system, which is configured for communication with a master-side console 1000 that is non-local or remote with respect to the actuation controller 700, the system endoscope 10, and hence the patient 5. The actuation controller 700 can (a) manipulate a set of robot arms 400 and corresponding end effectors in response to a surgeon's interaction with or manipulation of portions of the master-side console 1000, and (b) generate force feedback signals directed to the master-side console 1000, for instance, in a manner that is essentially identical, analogous, or generally analogous to that described in PCT Publication No. WO 2010/138083.

Aspects of Selectively Shape Lockable Primary Endoscope Probe Embodiments

FIG. 2 is a schematic illustration of a primary endoscope probe body 110 configured for selective or selectable shape locking in accordance with an embodiment of the present disclosure. In an embodiment, the primary endoscope probe body 110 carries a number of flexible cables 120 that are controllable or accessible beyond, near, or at the proximal end 102 of the primary endoscope probe 100, and which can be selectively or selectably tensioned or relaxed. In some embodiments, the cables 120 are coupled to actuated joints 150 carried within the endoscope probe body 110 (e.g., a first pair of cables 120a can be coupled to a first joint 150a; a second pair of cables 120b can be coupled to a second joint 150b that is distal to the first joint 150a; and a third pair of cables 120c can be coupled to a third joint 150c that is distal the second joint 150b). Such joints 150 are independently bendable, and can be independently controlled/controllable by way of the cables 120. More specifically, such joints 150 can be selectively or independently bent and locked into position by way of tensioning cables 120 coupled thereto (e.g., counterpart cables 120 corresponding to each joint 150, where a counterpart cables 120 can be tensioned or de-tensioned relative to each other) and maintaining the tension applied to the cables 120, in a manner readily understood by one having ordinary skill in the relevant art.

Additionally, each joint 150 corresponds to a given (e.g., predetermined) or distinct shape controllable or shape lockable section or segment along the primary endoscope probe body's length.

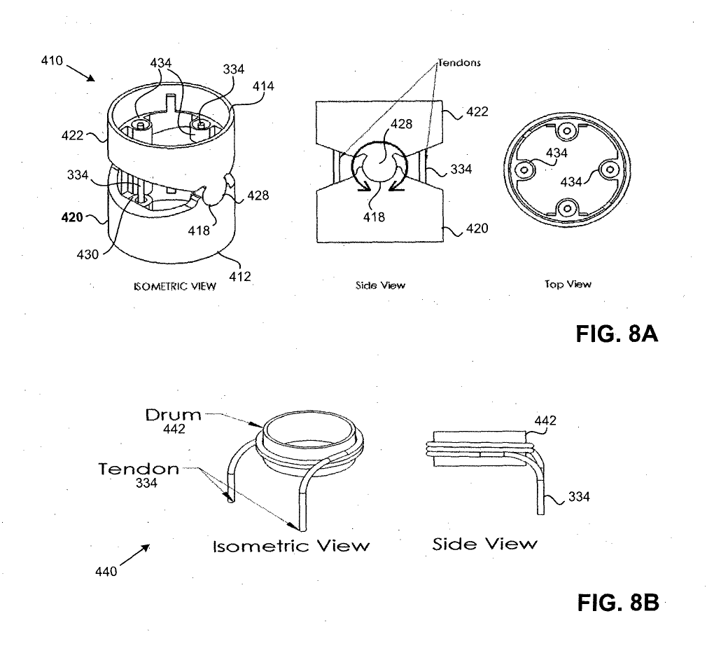

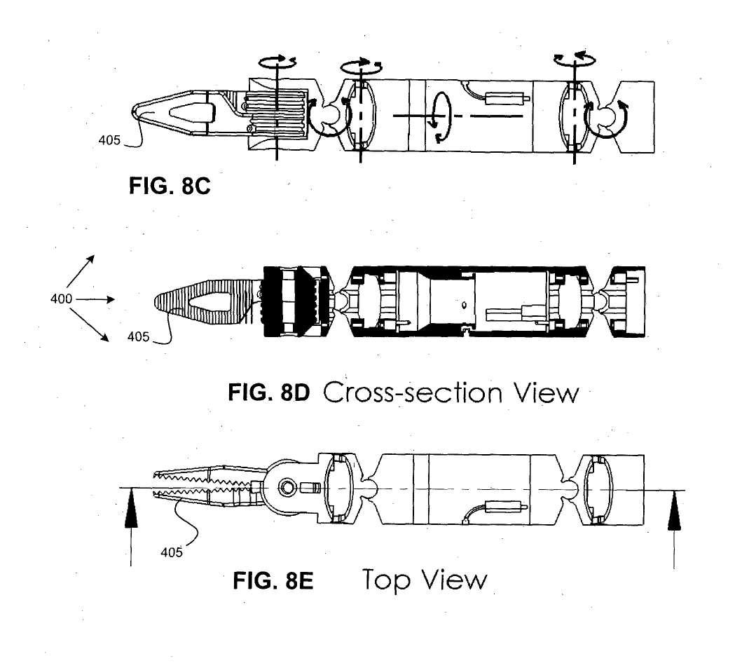

When the cables 120 are slack, substantially slack, loosened, or non-tensioned, the axial or longitudinal shape, profile, or orientation of the endoscope probe body 110 can shape-adapt or be changed (e.g., during primary endoscope probe navigation toward and into a target environment) in a manner essentially identical or analogous to that for a conventional flexible endoscope. Consequently, when the cables 120 are slack, substantially slack, or non-tensioned, the primary endoscope probe 100 can be inserted into an environment (e.g., a subject's body) and navigated therethrough in a manner identical or essentially identical to that for a conventional flexible endoscope. Once the primary endoscope probe 100 has reached an intended/desired or expected destination, or an intended/desired or expected intended/desired shape of the primary endoscope probe 100 has been attained within the environment in which the primary endoscope probe 100 resides, one or more portions or segments of the primary endoscope probe body 110 can be shape locked by way of applying tension to particular cables 120 (e.g., by way of pulling such cables 120) to thereby fix or lock the positional orientation of the joint(s) 150 corresponding to such tensioned cables 120, correspondingly fixing or locking the positional orientation of the endoscope probe body section or segment to which each joint 150 corresponds. Such tension upon the cables 120 can be maintained during a portion an endoscopic procedure or surgical intervention to maintain the primary endoscope probe 100 in its current shape and position, for instance, in a manner that conforms to the internal environment in which the primary endoscope probe 100 is disposed. When the primary endoscope probe 100 is to be withdrawn from the environment in which it is disposed, the cables 120 can be de-tensioned or relaxed, and the primary endoscope probe 100 can be removed in a manner identical or analogous to that for a conventional endoscope. In some embodiments, a joint 150 can have a structure that is substantially identical or analogous to a vertebra joint primitive 410 described below with reference to FIG. 8A, and the joint 150 can operate by way of cable tensioning in a manner that is identical or analogous to that for the vertebra joint primitive 410.

In another embodiment, actuated joints 150 can be omitted, in which case cables 120 of different lengths can be internally coupled to the primary endoscope probe body 110 (e.g., attached within or to the walls of the body 110). Once a desired primary endoscope probe body shape is attained (e.g., when the distal end 104 of the primary endoscope probe 100 is disposed at or proximate to an intended target environment), the cables 120 can be collectively tensioned, thereby stiffening or shape locking the longitudinal profile of the primary endoscope probe body 110 along or substantially along its entire length.

In still further embodiments, a primary endoscope probe body 110 can be selectively shape controlled/controllable/lockable using a combination of the aforementioned approaches, that is, by way of both (a) a set of cable-controlled actuated joints 150 and corresponding control cables 120, where each actuated joint 150 is disposed relative to a particular longitudinal position within the primary endoscope probe body 100; and (b) a set of cables 120 that is not coupled to actuated joints 150, where the cables 120 terminate at particular longitudinal distances along the primary endoscope probe body length.

Aspects of Primary/Secondary Endoscope Probe Embodiments

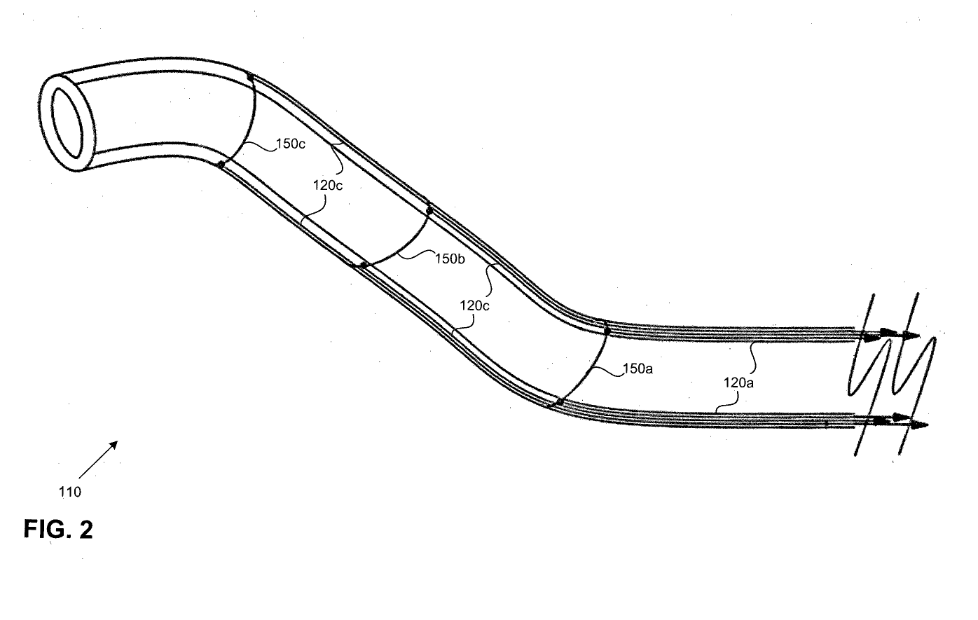

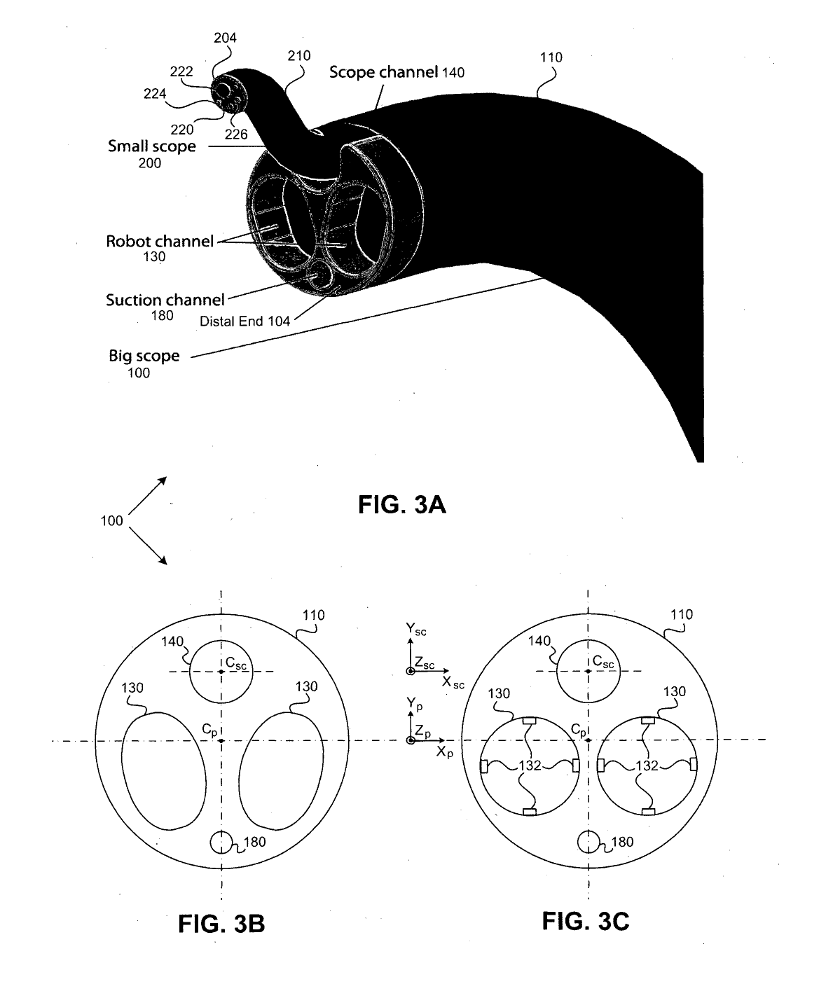

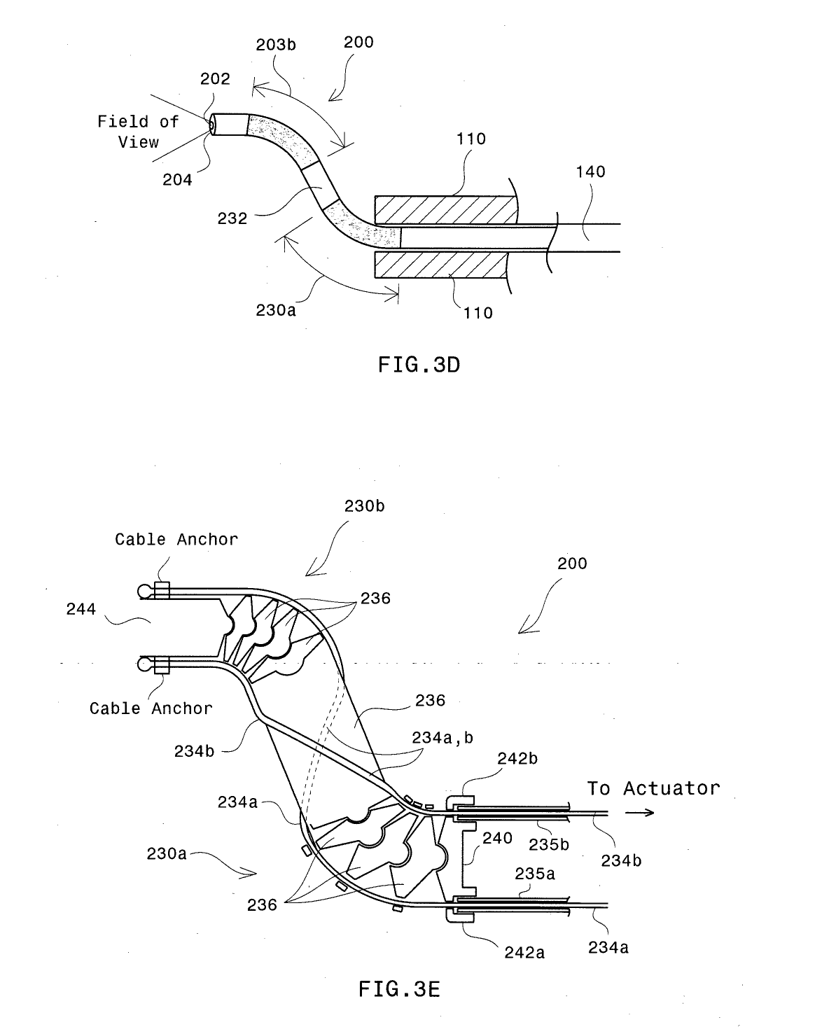

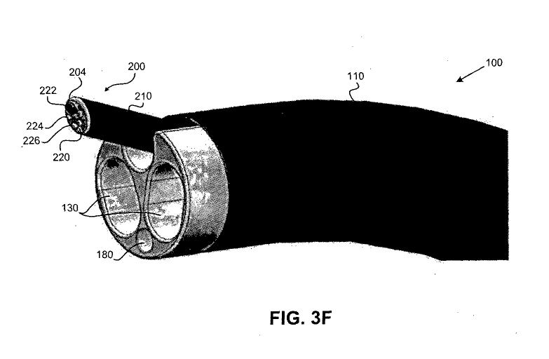

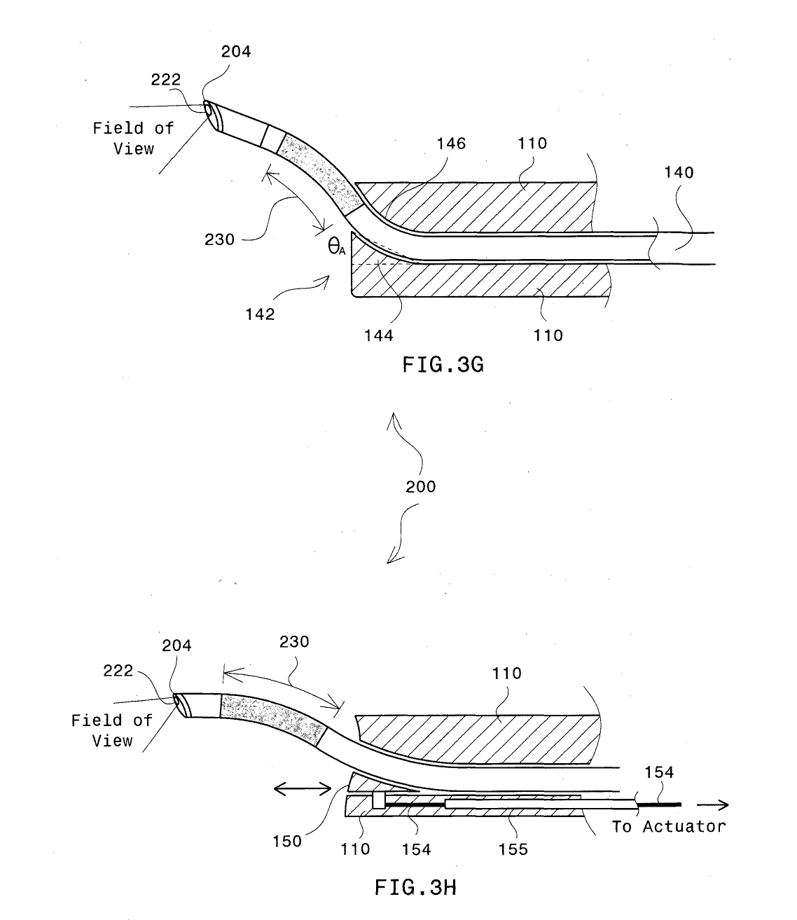

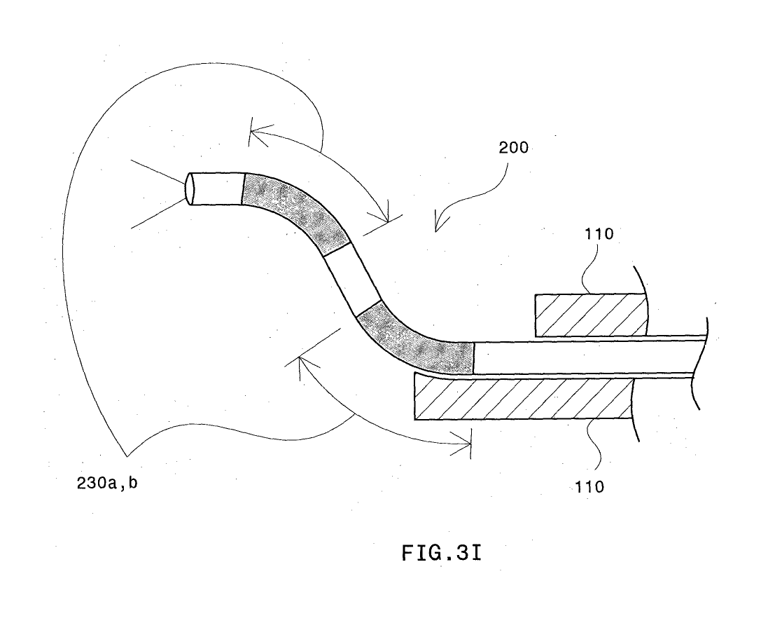

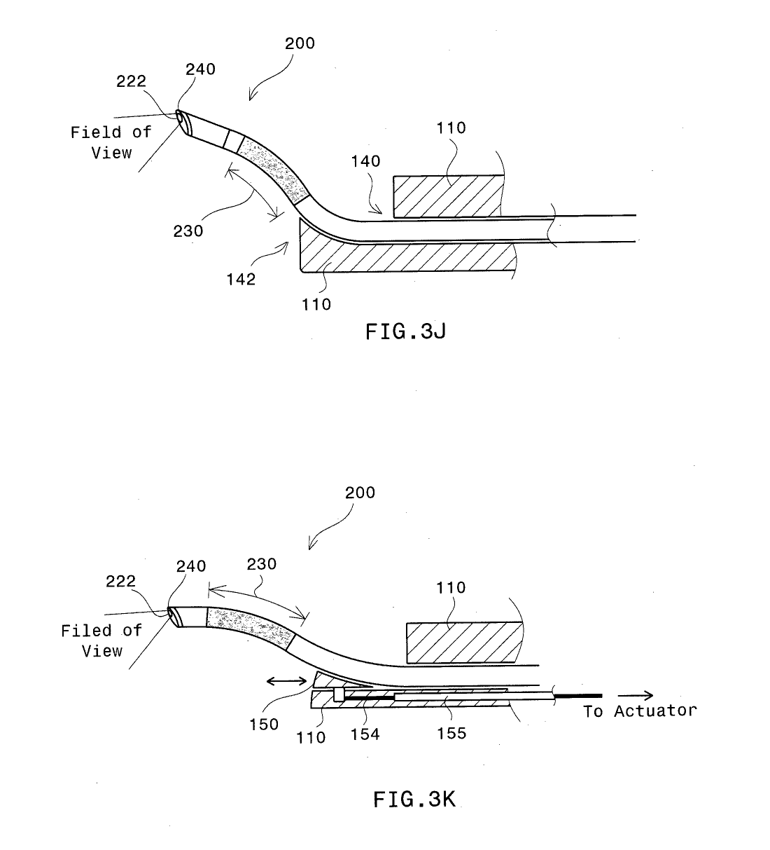

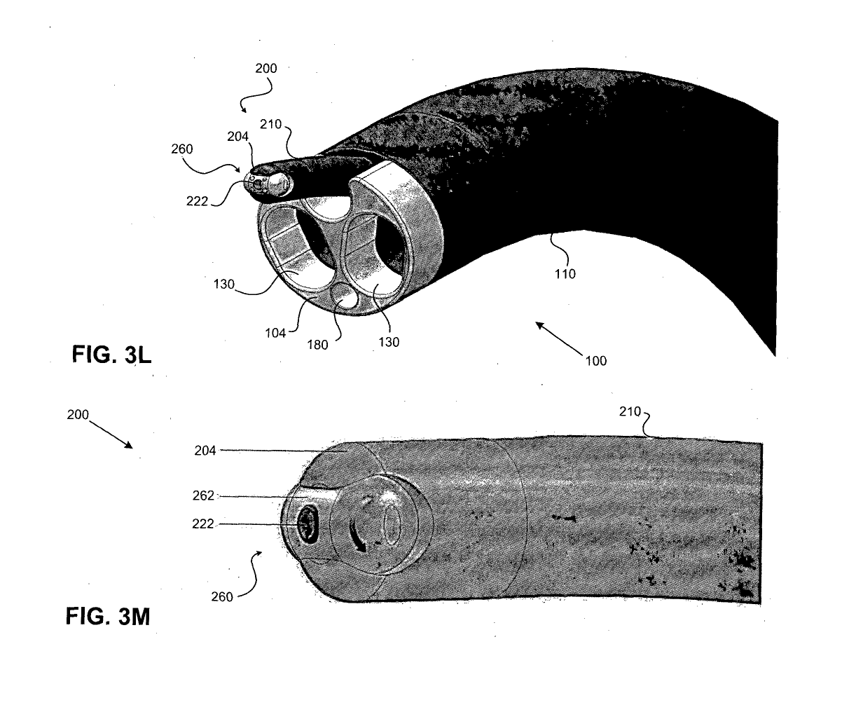

FIG. 3A is a schematic illustration of a primary endoscope probe 100 configured for carrying a secondary endoscope probe 200, such as an imaging and/or other type of endoscope (e.g., an ultrasound endoscope), in accordance with an embodiment of the present disclosure. As indicated above, the primary endoscope probe 100 includes a body 110 having a number of channels therein that extend from the primary endoscope probe's proximal end 102 to its distal end 104.

In an embodiment, the channels include (a) a set of tool channels 130a,b through which surgical tools and corresponding tool control and sensing elements, such as robot arms 400, end effectors, corresponding tendon-sheath drive elements, and any required electrical elements/connections, can be easily and reliably removably inserted (e.g., inserted and selectively withdrawn); (b) a secondary endoscope probe channel 140 through which a secondary endoscope probe 200 can be removably inserted; and (c) a number of adjunct endoscopy channels 180, such as a suction and/or an insufflation channel Each of such channels includes a corresponding opening at or proximate to the primary endoscope probe's distal end 104. More particularly, each tool channel 130 includes an opening through which a tool such as a robot arm 400 can extend and access a target anatomical environment, region, or tissue external to the primary endoscope probe 100; the secondary endoscope probe channel 140 includes an opening through which a secondary endoscope probe 200 can extend and access portions of the target anatomical environment, region, or tissue; and each adjunct channel includes an opening through which an adjunct endoscopy function can be provided proximate to or near the target anatomical environment, region, or tissue.

Portions of any given channel and its corresponding channel opening can exhibit essentially any type of cross sectional geometric profile, shape, or dimension for (i) accommodating one or more types of endoscopy tools/devices (e.g., robot arms 400 and corresponding end effectors), and (ii) facilitating reliable control of an endoscopy device provided by way of the channel, and reliable interaction of the endoscopy device with the target environment, region, or tissue in which the endoscopy device is disposed. For instance, one or multiple tool channels 130 can have a generally or somewhat circular, elliptical, or other type of cross sectional geometry.

Furthermore, one or more channels can include structural features, elements, or mechanisms therein that facilitate secure positioning/repositioning of endoscopy devices or tools carried thereby. For instance, a tool channel 130 can include a docking mechanism disposed therein, proximal or adjacent to the primary endoscope probe's distal end 104, which provides a support or brace element that enables (a) selective locking of a distal portion of a disposable actuation assembly 300 and/or a base portion of a robot arm 400 corresponding thereto to the brace element; (b) secure, predictable robot arm manipulation positioning during a surgical procedure; and (c) selective/selectable release of the robot arm 400 from the brace element such that the robot arm 400 can be readily withdrawn from the primary endoscope probe 100.

In various embodiments, the body 110 of the primary endoscope probe 100 has a cross sectional area or diameter (e.g., a distal end diameter) that larger or significantly larger than the cross sectional area or diameter of each tool channel 130, and which is significantly larger (e.g., several times larger) than the cross sectional area or diameter of the secondary endoscope probe channel 140. The tool channels 130 should be sufficiently large to accommodate the cross sectional areas of robot arms 400, various types of effectors that the robot arms 400 can carry, tendon-sheath drive elements, and any required electrical connections, subject to dimensional constraints imposed upon the overall acceptable cross sectional area of the primary endoscope probe 100, particularly in view of the cross sectional area of the secondary endoscope probe 200. Typically, the cross sectional area of the secondary endoscope probe channel 140 is smaller than the cross sectional area of each tool channel 130.

FIG. 3B is a front cross sectional view of a primary endoscope probe 110 configured for carrying a secondary endoscope probe 200 in accordance with an embodiment of the present disclosure. In various embodiments, a central or longitudinal axis of the primary endoscope probe 100 (e.g., a primary endoscope probe z-axis, Zₚ) can be defined which (a) extends parallel to or along a center or centroid (e.g., Cₚ) of the primary endoscope probe 100; and (b) is transverse or perpendicular to a cross sectional area of the primary endoscope probe 100, and hence extends through a plane defined by a primary endoscope probe x-axis Xₚ and y-axis Yₚ.

The secondary endoscope probe channel 140 is cooperatively disposed relative to the cross sectional area of the primary endoscope probe 100 such that the secondary endoscope probe 200 is aligned relative to the central axis of the primary endoscope probe 100. More particularly, a center, centroid, central, or longitudinal axis of the secondary endoscope probe channel 140 (e.g., a secondary endoscope probe channel z-axis, Z_{sc}) can be defined which (a) extends parallel to or along a center or centroid (e.g., C_{sc}) of the secondary endoscope probe channel 140; (b) is parallel to the primary endoscope probe central axis Zₚ; (c) is transverse or perpendicular to the primary endoscope probe cross sectional area, and hence extends through a plane defined by the primary endoscope probe x-axis Xₚ and y-axis Yₚ; and (d) is offset (e.g., vertically offset) from the primary endoscope probe's center or centroid Cₚ in a direction perpendicular to the primary endoscope probe's central axis Zₚ.

Thus, the secondary endoscope probe channel central axis Z_{sc} (a) resides in and extends through a common first plane relative to the primary endoscope probe central axis Zₚ toward, proximate to, or at the primary endoscope probe's distal end 104, but is (b) offset (e.g., vertically offset) from a second plane (e.g., a z-x plane) in which the primary endoscope probe center Cₚ resides, and from which the primary endoscope probe central axis Zₚ extends at the primary endoscope probe distal end 104.

In a manner analogous to that for the primary endoscope probe 100, a central or longitudinal axis can be defined for the secondary endoscope probe (e.g., a secondary endoscope probe z-axis, Zₛ), which (a) extends parallel to or along a center or centroid (e.g., Cₛ) of the secondary endoscope probe 200; and (b) is transverse or perpendicular to a cross sectional area of the secondary endoscope probe 200, and hence extends through in a plane defined by a secondary endoscope probe x-axis Xₛ and y-axis Yₛ. When the secondary endoscope probe 200 is carried by the secondary endoscope probe channel 140 of the primary endoscope probe 100 (e.g., such that as the secondary endoscope probe 200 extends proximate to and beyond the distal end of the primary endoscope probe 100, the majority of the secondary endoscope probe central axis Zₛ is parallel to the primary endoscope probe central axis Zₚ).

During deployment or navigation of the primary endoscope probe 110 toward, into, or to a target environment, a distal end 204 of the secondary endoscope probe 200 faces and is exposed/exposable to the target environment. When the secondary endoscope probe 200 is an imaging endoscope, such axial alignment of the secondary endoscope probe distal end 204 relative to the primary endoscope distal end 104 enables the imaging endoscope to capture a forward facing view at and/or beyond the distal end 104 of the primary endoscope probe 100, vertically offset from (e.g., “above” or “below,” in relative terms) the primary endoscope probe central axis Zₚ, for purpose of imaging the primary endoscope probe's navigation progress.

As indicated in FIG. 3A, portions of the secondary endoscope probe 200 can be configured for axially extending beyond the distal end 104 of the primary endoscope probe 100 into the target environment surrounding the distal end 104 of the primary endoscope probe 100, and possibly maneuvering within the target environment in one or more manners. The positioning or displacement of the secondary endoscope probe 200 beyond or away from the distal end 104 of the primary endoscope probe 100 can be controlled independent of and/or in addition to the positioning of the primary endoscope probe 100 within the target environment. For instance, after the distal end 104 of the primary endoscope probe 100 has been navigated to and is essentially stationary at an intended location within the target environment, the distal end 204 of the secondary endoscope probe 200 can be axially displaced (e.g., multiple centimeters) beyond the primary endoscope probe's distal end 104, in a direction parallel to the primary endoscope probe central axis Zₚ at the primary endoscope probe's distal end 104, by way of surge motion.

Additionally, in several embodiments, distal portions of the secondary endoscope probe 200 can be translated (e.g., one or more centimeters) toward or away from said primary endoscope probe central axis Zₚ by way of heave motion and/or possibly sway motion. As a result, near, proximate to, at, and/or beyond the secondary endoscope probe distal end 204, the secondary endoscope probe central axis Zₛ need not remain or be parallel to the primary endoscope probe central axis Zₚ because portions of the secondary endoscope probe 204 near, proximate to, and/or at the secondary endoscope probe distal end 204 can be selectively manipulated, positioned, or spatially disposed relative to the primary endoscope probe distal end 104.

The DOFs relative to which the secondary endoscope probe 200 can be positioned are intended to facilitate or enable selective and controlled/controllable positioning of the secondary endoscope probe 200 with respect to the DOFs relative to which the robot arm(s) 400 and corresponding effector(s) can interact with an anatomical site, structure, or tissue under consideration. As further described below, in several embodiments the distal end 204 of the secondary endoscope probe 200 is configured for anterograde and retrograde positioning relative to a spatial volume in which or target site at which the robot arm(s) 400 and effector(s) are positionable.

Also as indicated in FIG. 3A, in a number of embodiments the secondary endoscope probe channel 140 terminates before reaching the distal end 104 of the primary endoscope probe 100. That is, the opening of the secondary endoscope probe channel 140 is offset or set back a predetermined distance (for instance, at least approximately 0.2 cm, or between 0.1-20.0 cm, e.g., 0.1-15 cm, or 0.2-10 cm, or 0.5-5.0 cm) away from the distal end 104 of the primary endoscope probe 100, or offset/set back a predetermined percentage of the primary endoscope probe's length (for instance, up to 10%, 15%, or 20% of the primary endoscope probe's length, e.g., 0.1%-20%, 0.1%-15%, 0.2%-10%, or 0.2%-5% of the primary endoscope probe's length) away from the primary endoscope probe's distal end 104. Such positioning of the secondary endoscope probe channel's opening facilitates enhanced translation of the distal end 204 of the secondary endoscope probe 200 transverse to the central axis Zₚ of the primary endoscope probe 100 under conditions of small or minimal displacement of the secondary endoscope probe's distal end 204 in a direction parallel to the primary endoscope probe central axis Z. In other words, such positioning of the secondary endoscope probe channel's opening facilitates greater heave displacement (and/or possibly greater surge displacement in some embodiments) of distal portions of the secondary endoscope probe 200 when surge displacement of the distal end 204 of the secondary endoscope probe 200 beyond the distal end 104 primary endoscope probe 100 is small or minimal.

Consequently, when the secondary endoscope probe 200 includes an imaging/image capture device, the distal end 204 of the secondary endoscope probe 200 can be vertically displaced from the primary endoscope probe central axis Zₚ in a manner that increases the portion of the target environment that falls within the imaging device's field of view. The imaging device can thus more effectively capture images corresponding to the spatial volume in which the robot arm(s) 400 and effector(s) are disposed/disposable, including “top down” images of the robot arm(s) 400 and effector(s) as they are manipulated. Furthermore, the imaging device can thus capture images at or very near the distal end of the primary endoscope probe 100, including images of the robot arm(s) 400 and/or end effectors in situations in which the robot arm(s) and/or end effector(s) are operating at or very near (and possibly even slightly proximal to) the distal end 104 of the primary endoscope probe 100.

Additionally, such an offset of the secondary endoscope probe channel's distal opening away from the distal end 104 toward the proximal end 102 of the primary endoscope probe can selectively enable operation of the secondary endoscope probe 200 within a spatial volume that is at or at least slightly proximal to the distal end 104 of the primary endoscope probe 100. For instance, when the secondary endoscope probe 200 includes an imaging device, the imaging device can capture images not only beyond the distal end of the primary endoscope probe 100 (e.g., when the secondary endoscope probe 200 is surged past the distal end 104 of the primary endoscope probe 100), but also images at and at least slightly proximal to the distal end 104 of the primary endoscope probe 100, even when the primary endoscope probe 100 has been positioned or “parked” at an intended destination, without requiring primary endoscope probe repositioning. The imaging device can correspondingly capture images that can visually indicate whether an environment at or at least slightly proximal to the distal end 104 of the primary endoscope probe 100 is stable or has been affected by a procedure that is occurring beyond the distal end 104 of the primary endoscope probe 100, in a spatial region in which the robot arm(s) 400 and corresponding end effector(s) are operating, while only slightly or insignificantly affecting or distorting the environment at and around which the distal end 104 of the primary endoscope probe 100 resides.

In general, a minimal proximal offset of the secondary endoscope probe channel's distal opening away from the distal end 104 of the primary endoscope probe 100 should at least facilitate the capture of top down images of the robot arm(s) 400 and end effector(s) at or very near the distal end 104 of the primary endoscope probe 100; and a maximal proximal offset of the secondary endoscope probe channel's distal opening away from the distal end 104 of the primary endoscope probe 100 should avoid requiring that the secondary endoscope probe 200 is configured for an excessive or extreme amount of surge motion in order to reach the distal end 104 of the primary endoscope probe 100, and should ensure that the secondary endoscope probe 200 and the primary endoscope probe 100 together remain a closely integrated, highly compact unit that can be readily navigated within an environment (e.g., where the leading/most distal surface for such navigation is the distal end 104 of the primary endoscope probe 100 from which the robot arm(s) 400 and end effector(s) emerge).

In various embodiments in which the secondary endoscope probe 200 includes or is an imaging endoscope of a type described herein with reference to FIGS. 3D-3M, the proximal offset of the secondary endoscope probe channel 140 away from the distal end 104 of the primary endoscope probe 100, possibly further in combination with additional structural features described herein such as a tapered section 142 or a ramp structure 150 as described below with reference to FIGS. 3G, 3H, and 3K, can significantly or greatly enhance the ability of the imaging endoscope 200 to selectively capture images across or within a image capture volume or range that includes spatial regions beyond, very near, at, and possibly proximal to the distal end 104 of the primary endoscope probe 100.

FIG. 3C is a front cross sectional view of a primary endoscope probe 110 configured for carrying a secondary endoscope probe 200 in accordance with another embodiment of the present disclosure. In several embodiments, one or more primary endoscope probe tool channels 140 includes a set of guide, retention, bracing and/or securing structures or elements 132 (e.g., track, rail, displacement limitation, and/or displacement stop members) proximate to and/or at its distal end 104. Such guide/bracing/retention/ securing elements 132 are configured for mating engagement and selective disengagement, which can include locking/lockable engagement (e.g., keyed engagement), with counterpart structures or elements (e.g., apertures, recesses, channels, receivers, and/or notched or keyed collar elements) carried by portions of a disposable actuation assembly 300 that are intended to reside proximate to and/or at the primary endoscope probe distal end 104 when the robot arm(s) 400 and effector(s) are deployed and ready for manipulation or use within a target environment external to the distal end 104 of the primary endoscope probe 100.

When such guide/retention elements 132 are matingly engaged with and/or captured by counterpart structures or elements carried by a disposable actuation assembly 300, the disposable actuation assembly 300 and the robot arm 400 and effector 405 supported thereby can be defined to reside in a deployment/deployed position. In some embodiments, following insertion of a disposable actuation assembly 300 to a given axial depth within the primary endoscope probe 100, further axial displacement of the disposable actuation assembly 300 is prevented unless the primary endoscope probe guide/retention/securing elements 132 and their counterpart elements carried by the disposable actuation assembly 300 are appropriately aligned. Rotation of the disposable actuation assembly 300 in a predetermined direction relative to the primary endoscope probe 100 can place the disposable actuation assembly 300 in the deployment/deployed position, thereby enabling secure retention of the disposable actuation assembly 300 within the primary endoscope probe 100. Correspondingly, after the disposable actuation assembly 300 is in the deployment/deployed position, rotation of the disposable actuation assembly 300 in an opposite direction can facilitate removal or withdrawal of the disposable actuation assembly 300 from the primary endoscope probe 100.

In multiple embodiments, the primary endoscope probe guide elements 132 and counterpart channel elements carried by a disposable actuation assembly 300 are configured for providing an axial or longitudinal displacement distance (e.g., multiple or several centimeters, for instance, approximately 3-5 centimeters, or between approximately 5-10 centimeters, or up to about 10-12 centimeters) along or through which the disposable actuation assembly 300 can be selectively axially or longitudinally translated or displaced relative to the primary endoscope probe central axis Zₚ, such as by way of a set of translation mechanism actuators. Thus, when a disposable actuation assembly 300 exists in a deployment/deployed position, the disposable actuation assembly 300 can be axially translated within or across a maximum axial translation distance, while the disposable actuation assembly 300 remains securely retained within the primary endoscope probe 100. Consequently, a robot arm 400 and an effector 405 carried thereby can be selectively axially translated relative to the distal end 104 of the primary endoscope probe 100, for instance, to facilitate or enable an intended axial positioning of the robot arm 400 and effector 405 within the target environment.

For any given type of secondary endoscope probe 200 under consideration, the secondary endoscope probe's positioning/maneuvering capabilities depend upon the secondary endoscope probe's intended function(s), and the secondary endoscope probe's physical construction. Various non-limiting representative secondary endoscope probe embodiments in which the secondary endoscope probe 200 includes, is based upon, or is an imaging endoscope are provided hereafter with respect to FIGS. 3A-3M. In each of such representative embodiments, the imaging endoscope 200 includes a body 210 having a proximal portion, segment, section, or end that is coupled/couplable to the endoscopist interface 30, and which extends along an imaging endoscope length to a distal end 204 that is independently or separately positionable, manipulable, or controllable relative to the distal end 104 of the primary endoscope probe 100. The imaging endoscope 200 includes a face 220 disposed at its distal end 204, which carries an image capture module or camera module 222, a set of illumination sources (e.g., LEDs, optical fibers, and/or lens elements) 224, and possibly one or more adjunct endoscopy elements or devices 226, such as an insufflation aperture, in a manner understood by one of ordinary skill in the relevant art. Furthermore, each such imaging endoscope 200 is configured for at least surge displacement relative to the primary endoscope probe 110.

As depicted in FIG. 3A, and as further detailed in FIGS. 3D and 3E, in some embodiments the imaging endoscope 200 is an S-bend endoscope, which includes a first controllable region 230a, a second controllable region 230b, and a substantially rigid section 232 disposed therebetween. More particularly, the rigid section 232 is distally disposed relative to the first controllable region 230a, and the second controllable region is distally disposed relative to the rigid section 232. In several embodiments, at least substantial portions of the first controllable region 230a, and hence the entire rigid section 232 and the second controllable region 230b, can surge beyond the distal terminus of the S-bend endoscope channel 140. Each of the first and second controllable regions 230a,b is configured for enabling heave displacement, such that the S-bend imaging endoscope 200 can be selectively and controllably manipulated to capture anterograde and retrograde views of robot arm/effector operation within the target environment. In a number of embodiments, manipulation of the controllable regions 230a,b occurs by way of cable elements configured for manipulating adjacently stacked vertebrae-type joint elements.

FIG. 3E is a schematic illustration of cables 234 and vertebrae 236 within an S-bend imaging endoscope 200 in accordance with an embodiment of the present disclosure. In an embodiment, each of the S-bend endoscope's controllable regions 230a,b includes a set of vertebrae 236, each of which can be displaced relative to another vertebra 236 by way of first and second cables 234a,b. The number and/or thicknesses (e.g., defined relative to the S-bend imaging endoscope's central axis) of the vertebra 236 within the first controllable region 230a can be the same as or different than that of the second controllable region 230b, depending upon embodiment details.

Each vertebra 236 is configured for mating engagement with and central pivotable displacement about an adjacent vertebra 236, such as by way of protrusions and recesses that form ball-and-cup/ball-and-socket pivots or pivot points, which are centrally disposed relative to each vertebral transverse extent or cross sectional area. Each vertebra 236 has an outer or peripheral surface, which includes a first outer surface site that is closest to the primary endoscope probe's longitudinal axis, and a second outer surface site that is opposite to (e.g., directly opposite to or across from) the first outer surface site. Thus, each vertebra's first outer surface site and second outer surface site are on opposite sides of the S-bend imaging endoscope's longitudinal axis.

Within the first controllable region 230a, each vertebra's first outer surface site is coupled or linked to the first cable 234a, and each vertebra's second outer surface site is coupled or linked to the second cable 234b. Thus, the first and second cables 234a,b are disposed on opposite sides of any given vertebra 236. In an analogous yet converse manner, within the second controllable region 230b, each vertebra's first outer surface site is coupled or linked to the second cable 234b, and each vertebra's second outer surface site is coupled or linked to the first cable 234a. The first and second cables 234a,b cross each other within the rigid section 232 to facilitate such vertebral couplings or linkages within the first and second controllable regions 230a,b.

The first controllable region 230a additionally includes a reference proximal vertebra 240 relative to which the vertebra 236 within the first controllable region 230a (and hence the vertebra 236 within the second controllable region 230b) are distally disposed; and the second controllable region 230b includes a reference distal vertebra 244 relative to which the vertebra 236 within the second controllable region 230b (and hence the vertebra 236 within the first controllable region 230a) are proximally disposed. The position of the reference proximal vertebra 240 within the S-bend imaging endoscope 200 is fixed or anchored at a predetermined location of the S-bend imaging endoscope's length; and the position of the reference distal vertebra 244 is fixed or anchored at a predetermined location near or adjacent to the S-bend imaging endoscope's distal end 204. Each of the reference proximal and reference distal vertebra 240, 244 includes a periphery or outer surface.

The reference proximal vertebra 240 includes a centrally disposed protrusion or recess that is configured for mating engagement with a counterpart centrally disposed recess or protrusion, respectively, of an adjacent vertebra 236 within the first controllable region 230a, such that this adjacent vertebra 236 can pivot relative to the reference proximal vertebra 240. Similarly, the reference distal vertebra 244 includes a centrally disposed protrusion or recess that is configured for mating engagement with a counterpart centrally disposed recess or protrusion, respectively, of an adjacent vertebra 236 within the second controllable region 230b to facilitate pivotable displacement of the vertebra 236 relative to the reference distal vertebra 244.

The reference proximal vertebra 240 includes a first receiving structure 242a and a second receiving structure 242b, each of which is internal yet proximate to its periphery. The first receiving structure 242a is disposed closest to the primary endoscope probe's central or longitudinal axis, and the second receiving structure 242b is disposed opposite to (e.g., directly opposite to or across from) the first receiving structure 242a. Thus, the first and second receiving structures 242a,b are on opposite sides of the S-bend imaging endoscope's central axis.

The first receiving structure 242a is configured for receiving a first sheath 235a within which the first cable 234a is carried along portions (e.g., the majority) of the S-bend imaging endoscope's length, such that the first cable 234a can extend to and beyond the S-bend imaging endoscope's proximal end and be coupled to the actuation controller 700. The first receiving structure 242a provides an abutment against which a distal end of the first sheath 235a is disposed/disposable, where such abutment includes an opening through which the first cable 234a can pass and extend towards the S-bend endoscope's distal end 204.

Similarly, the second receiving structure 242b is configured for receiving a second sheath 235b within which the second cable 234b is carried along portions (e.g., the majority) of the S-bend imaging endoscope's length, such that the second cable 234b can extend to and beyond the S-bend imaging endoscope's proximal end and be coupled to, the actuation controller 700. The second receiving structure 242b provides an abutment against which a distal end of the second sheath 235b is disposed/disposable, where such abutment includes an opening through which the second cable 234b can pass and extend towards the S-bend endoscope's distal end 204.

In a manner analogous to that previously described, the reference distal vertebra 244 includes an outer surface having a first outer surface site that is closest to the primary endoscope probe's longitudinal axis, and a second outer surface site that is opposite to the first outer surface site. Thus, the reference distal vertebra's first and second outer surface sites are on opposite sides of the S-bend imaging endoscope's central axis. Furthermore, the reference distal reference vertebra's first and second outer surface sites are near the S-bend imaging endoscope's distal end 204. The reference distal vertebra's first and second outer surface sites serve as anchor points for the cables 234. More particularly, due to the aforementioned cable crossover within the rigid section 232, the reference distal vertebra 244 provides an anchor point for the first cable 234a at its second outer surface site, and an anchor point for the second cable 234b at its first outer surface site. That is, the first and second cables 234a,b terminate and are anchored at the reference distal vertebra's second and first outer surface sites, respectively.

As a result of (a) the cable-to-vertebra couplings or linkages within the first and second controllable regions 230a,b; and (b) the cable crossover within the rigid section 232, a pulling force selectively or preferentially applied to one of the first and second cables 234a,b while the other of the first and second cables 234a,b remains accommodatively, responsively, or proportionately counter-tensioned, negatively tensioned, or relaxed causes vertebrae 236 within each of the first and second controllable regions 230a,b to pivot about vertebral pivot points such that vertebrae 236 within the first controllable region 230a pivot in a first flex direction, and vertebrae 236 within the second controllable region 230b pivot in a second flex direction that is opposite to the first flex direction. That is, the vertebrae 236 within the first controllable region 230a pivot in an opposite direction relative to the vertebrae 236 within the second controllable region 230b, such that the first and second controllable regions 230a,b counterflex relative to each other. Counterflexion of the vertebrae 236 in the first and second controllable regions 230a,b can occur substantially simultaneously.

For instance, a pulling force applied to the second cable 234b causes the first controllable region 230a to flex in a manner that vertically displaces the rigid section 232 and the second controllable region 230b away from each of the S-bend imaging endoscope's central axis and the primary endoscope probe's central axis. Moreover, maintaining or increasing this pulling force causes the second controllable region 230b to flex in a manner that bends the distal end 204 of the S-bend imaging endoscope 200 toward the primary endoscope probe's central axis, thereby positioning the field of view of the S-bend imaging endoscope's camera module 222 relative to a portion of the spatial region beyond the distal end 104 of the primary endoscope probe 100 through which the primary endoscope probe's central axis extends.

Thus, such counterflexion of the first and second controllable regions 230a,b results in (a) heave displacement of the S-bend imaging endoscope's camera module 222 away from the primary endoscope probe's central axis by way of vertebral motion within the first controllable region 230a, such that the camera module 222 is disposed above the primary endoscope probe's central axis; and (b) orientation of the camera module 222 such that the camera module's field of view is directed toward primary endoscope probe's central axis, by way of vertebral motion within the second controllable region 230b. This counterflexion positions the camera module 222 above the robot arm(s) 400 and corresponding end effector(s) disposed beyond the distal end 104 of the primary endoscope probe 100, in a manner that facilitates or enables the capture of anterograde and retrograde images of the robot arm(s) 400 and end effector(s) within a spatial volume within which or target site at which the robot arm(s) 400 and end effectors can operate.

The range of heave displacement across which the S-bend imaging endoscope's camera module 222 can be displaced, and the extent of anterograde/retrograde positioning of the camera module's field of view, can be controlled by way of selective application of pulling forces to the first and second cables 230a,b, in a manner that will be readily understood by one of ordinary skill in the art. Similarly, the appropriate application or release of pulling forces can result in (a) a re-alignment of the S-bend imaging endoscope's face 222 such that the central axis of the S-bend imaging endoscope 200 is approximately normal to the face 222; and (b) the withdrawal or retraction of the S-bend imaging endoscope's camera module 222 toward, to, or into the secondary endoscope probe channel 140, in a manner that will also understood by one of ordinary skill in the relevant art.

In a number of embodiments, the first sheath 235a (carrying the first cable 234a) and the second sheath 235b (carrying the second cable 234b) are carried within a disposable actuation assembly 300, which can be coupled to the actuation controller 700 by way of quick release interfaces 500, 600 as further detailed below. Depending upon embodiment details, the actuation controller's application or delivery of pulling forces to the first and second cables 234a,b can be managed or controlled by way of one or more imaging endoscope control elements (e.g., knobs or levers) carried by the endoscopist interface 30, and/or corresponding control elements or control functionality provided by the master-side console 100. Consequently, in a number of embodiments, a surgeon operating the master controller or console 1000 can control the positioning of the S-bend imaging endoscope 200, for instance, by way of a joystick, foot pedal controls, voice commands; and/or gesture recognition (e.g., hand gesture/motion, and/or head gesture/motion recognition), in association with their manipulation of the robot arm(s) 400 and corresponding end effector(s); or an endoscopist can control the positioning of the S-bend imaging endoscope 200. Such surgeon control/endoscopist control of the S-bend imaging endoscope 200 can occur in a selectable manner, for instance, corresponding to default endoscopist control, with surgeon override control.

FIGS. 3F-3H are schematic illustrations of a primary endoscope probe 100 configured for carrying a bevel tip imaging endoscope 200 in accordance with an embodiment of the present disclosure. In an embodiment, the bevel tip imaging endoscope 200 includes a face 220 that is positioned at a non-normal angle relative to the bevel tip imaging endoscope's central or longitudinal axis, in a manner that inherently disposes the field of view of a camera module 222 carried by the face 222 toward the central or longitudinal axis of the primary endoscope probe 100. Consequently, the camera module's field of view is inherently disposed toward the primary endoscope probe's central axis, and hence is angularly biased for capturing images within portions of a spatial volume within which a set of robot arms 400 and corresponding end effectors can operate, as will be readily understood by one of ordinary skill in the relevant art.

The bevel tip imaging endoscope 200 is configured for surge displacement relative to the terminus of the secondary endoscope probe channel 140. In an embodiment, the distal end 204 of the bevel tip imaging endoscope 200 is also configured for heave displacement relative to the bevel tip imaging endoscope's central axis, and hence the primary endoscope probe's central axis, by way of (a) a tapered section 142 along a distal portion of the secondary endoscope probe channel 140 and/or along a distal segment of the primary endoscope probe body 110 along which the imaging endoscope 200 can be displaced by way of surge motion; and (b) a single controllable region 230 within the bevel tip imaging endoscope 200, which can be articulated in a manner that facilitates the capture of anterograde and retrograde images of robot arm(s) 400 and end effector(s) relative to a set of target sites beyond the distal end 104 of the primary endoscope probe 100.

As shown in FIG. 3G, in an embodiment a tapered section 142 of the secondary endoscope probe channel 140 includes a lower taper member 144 and an upper taper member 146, where the lower taper member 144 resides closer to the primary endoscope probe's central axis than the upper taper member 146, which is opposite to the lower taper member 144. Taken together, the lower taper member 144 and the upper taper member 146 form an arc, curve, or bend along distal portions of the secondary endoscope probe channel 140, which progressively disposes increasingly distal portions of the secondary endoscope probe channel 140 further away from the primary endoscope probe's central axis. The curve provided by the lower and upper taper members 144, 146 vertically offsets or elevates the secondary endoscope probe channel's terminal opening by a predetermined articulation angle θ_{A} defined relative to a horizontal or longitudinal distance (e.g., parallel to the secondary endoscope probe channel's central or longitudinal axis) across which the lower and upper taper members 144, 146 begin and terminate. In various embodiments, the magnitude of θ_{A} depends upon an as-manufactured amount of curvature provided by the lower and upper taper members 144, 146 along an as-manufactured horizontal or longitudinal distance over which each of the lower and upper taper members 144, 146 exist.

As the distal end 204 of the bevel tip imaging endoscope 200 is surged toward, to, and beyond the secondary endoscope probe channel's terminus, the lower taper member 144 and the upper taper member 146 guide or direct distal portions of the bevel tip imaging endoscope 200 along the tapered section's curve, thereby displacing the distal end 204 of the bevel tip imaging endoscope 200 through the articulation angle θ_{A} in a direction away from the primary endoscope probe's central axis, and elevating the bevel tip endoscope's camera module 222 relative thereto. The curve provided by the lower and upper taper members 144, 146 thus effectively heaves the distal end 204 of the bevel tip imaging endoscope 200 as the bevel tip imaging endoscope 200 is surged.

As shown in FIG. 3H, in an embodiment the lower taper member 144 provided by the tapered section 142 is displaceable, such as by way of a ramp structure 150 coupled or linked to a cable 154 (e.g., a cable 154 that can be configured in a manner substantially identical to a Bowden cable; or a cable 154 that wraps around a wheel or pulley) that is carried by a corresponding sheath 155, and which is coupled/couplable to the actuation controller 700. The ramp structure 150 can be translated parallel to the secondary endoscope probe channel's central axis in response to forces applied to the cable 154. As a result, the horizontal or longitudinal lower taper member distance relative to which the taper angle θ_{A} is defined can be adjusted or modified, thereby adjusting or modifying the heave distance across which the bevel tip endoscope camera module 222 can be elevated.

The bevel tip imaging endoscope's controllable region 230 can have an internal structure that is essentially identical, analogous, or generally analogous to that described above for the S-bend imaging endoscope's second controllable region 230b. For instance, the bevel tip imaging endoscope's controllable region 230 can include a number of vertebra 236 that can be pivoted relative to each other by way of first and second cables 234a,b. The vertebra 236 can be disposed between a reference proximal vertebra 240 and a reference distal vertebra 244 in a manner analogous or generally analogous to that described above. The selective application of pulling forces to the first and second cables 234a,b can selectively orient the bevel tip imaging endoscope's camera module 222 such that its field of view can capture anterograde and retrograde images of robot arms 400 and end effectors.

In several embodiments, the primary endoscope probe body 110 can be structured in a manner that facilitates selective manipulation/positioning of the secondary endoscope probe 200 in particular DOFs, for instance, in a manner indicated in FIGS. 31-3K for distal portions of the primary endoscope probe body 110, as will be understood by one of ordinary skill in the relevant art.

FIG. 3L is a schematic illustration of a primary endoscope probe 100 configured for carrying an imaging endoscope 200 having a pivotable or rotatable image capture module or camera assembly 260, and FIG. 3M is a schematic illustration showing particular aspects of such an imaging endoscope 200 and rotatable camera assembly 260 in accordance with an embodiment of the present disclosure. As indicated in FIG. 3L, the imaging endoscope 200 is configured for at least surge displacement along the imaging endoscope's central axis (and correspondingly, along the primary endoscope probe's central axis). In certain embodiments, the imaging endoscope 200 can additionally be configured for heave and/or sway displacement. For instance, the imaging endoscope 200 can be configured for heave displacement by way of a secondary endoscope probe channel 140 having a tapered section 142, in a manner analogous to that describe above with reference to FIGS. 3F and/or 3G.

In an embodiment, the rotatable camera assembly 260 includes a rotatable housing 262 that carries a camera module 222. The rotatable housing 262 and the distal end 204 of the imaging endoscope 200 are configured for form fitting mating engagement with each other in a manner that facilitates or enables pivotable motion of the rotatable camera module 222 about an axis of rotation transverse to the imaging endoscope's central axis. In several embodiments, the rotatable housing 262 includes an outer surface that carries an external or distal portion of the camera module 222 (e.g., a lens element), and the distal end 204 of the imaging endoscope 200 includes a socket or cup in which portions of the rotatable housing 262 can be retained, yet pivotably displaced about the axis of rotation. Depending upon embodiment details, selective rotational displacement of the rotatable housing 262 can occur by way of a set of cables, or a micromotor carried within the imaging endoscope 200.

Absent any rotation or pivotal displacement of the rotatable housing 262, the camera module 222 can be oriented in accordance with a default forward view, such that the imaging endoscope's central axis extends through a center or centroid of the camera module's field of view and the camera module 222 can capture images within a spatial volume through which the imaging endoscope's central axis extends, directly beyond the imaging endoscope's distal end 204.

Simultaneous with selective/selectable rotation of the rotatable housing 262 about its axis of rotation, the camera module's field of view is rotated, pivoted, or directed toward or away from the primary endoscope probe's central axis. As a result, the camera module 222 can be rotatably displaced such that the camera module's field of view can selectively capture anterograde and retrograde images of the robot arm(s) and corresponding end effector(s) with respect to target sites at or along which the robot arm(s) and end effector(s) can operate within a spatial volume through which the primary endoscope probe's central axis extends.

As an alternative to the foregoing, in certain embodiments an imaging endoscope 200 having a rotatable camera assembly 260 such as that described with reference to FIGS. 3L and 3M can be used independent or exclusive of a primary endoscope probe 100 configured for carrying such an imaging endoscope 200. For instance, a conventional imaging endoscope can be modified or adapted at its distal end to carry a rotatable camera assembly 260 in accordance with an embodiment of the present disclosure, and the modified conventional imaging endoscope can be inserted into a patient 5 in association with a conventional endoscopic imaging procedure the need not or does not involve the manipulation of a set of robot arms and end effectors.

In certain further embodiments in accordance with the present disclosure, the distal end 104 of the primary endoscope probe 100 can be beveled or tapered at a predetermined angle (e.g., in a manner analogous to the face of the bevel tip imaging endoscope 200 described above. An upper portion of the primary endoscope probe's tapered distal end 104 can correspond to or include the secondary endoscope probe channel's distal opening; and/or the upper portion of the primary endoscope probe's tapered distal end 104 can carry a rotatable/pivotable camera module 260. A set of robot arms 400 and corresponding end effectors can extend beyond the distal end 104 of the primary endoscope probe 100, below the secondary endoscope probe channel's distal opening and/or the rotatable/pivotable camera module 260.

In various embodiments, significant portions of or substantially an entire secondary endoscope probe 200 can be inserted into and withdrawn from the primary endoscope probe 100. For instance, in essentially any of the foregoing embodiments described above in relation to FIGS. 3A-3M, one or more portions of the imaging endoscope 200 can be based upon or have a structure that is substantially identical to a conventional imaging endoscope, and the imaging endoscope 200 can be selectively inserted into and withdrawn from the primary endoscope probe 100 in a manner essentially identical or analogous to that for the insertion and withdrawal of tools from an endoscope tool channel, in a manner understood by one of ordinary skill in the relevant art. Furthermore, as previously indicated, actuatable elements (e.g., cables 234 and vertebrae 236) within an imaging endoscope 200 can be coupled to the actuation controller 700 by way of a disposable actuation assembly 300. In such embodiments, substantially or essentially the entire imaging endoscope 200 can be carried by a disposable actuation assembly 300; or the disposable actuation assembly 300 can proximally extend from the imaging endoscope 300 toward, through, and beyond the endoscopist interface 30. The disposable actuation assembly 300 can correspondingly be inserted into and withdrawn from the primary endoscope probe 100, thereby inserting the imaging endoscope 200 into and withdrawing the imaging endoscope 200 from the primary endoscope probe 100.

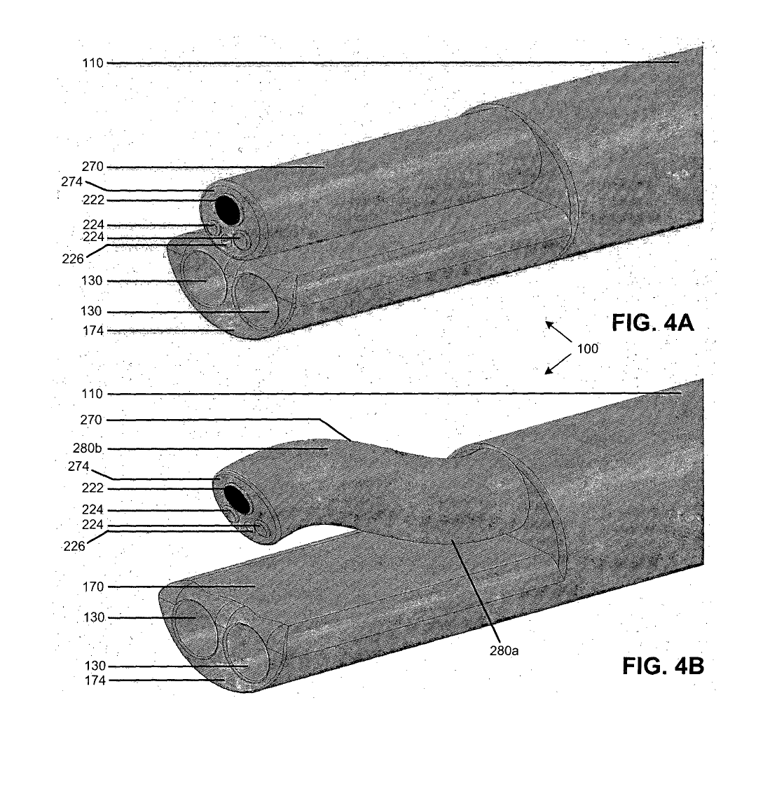

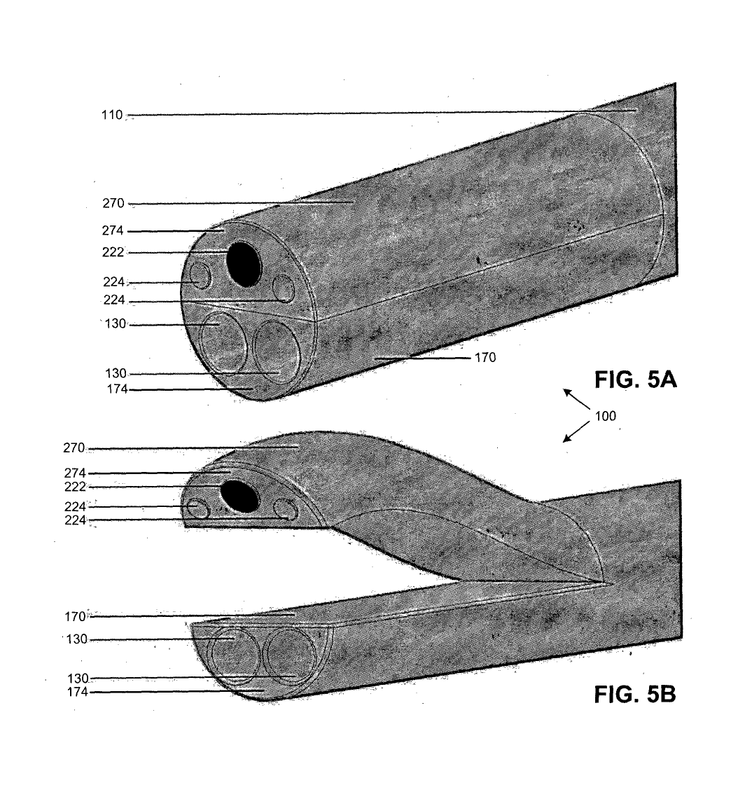

Beyond the foregoing, other embodiments in accordance with the present disclosure can include a secondary endoscope segment that forms a distal portion of the primary endoscope probe 100, as described hereafter with respect to non-limiting representative embodiments shown in FIGS. 4A-5B.

FIGS. 4A and 4B are schematic illustrations of a primary endoscope probe 100 that includes a secondary probe member 270 in accordance with an embodiment of the present disclosure. In an embodiment, the primary endoscope probe body 110 maintains a uniform or substantially uniform external or exterior profile along the majority of its length. However, near or generally near the distal end 104 of the primary endoscope probe 100, the primary endoscope probe body 110 is divided or segregated into a secondary probe member 270 which is distinct/distinguishable and selectively separable and manipulable from a tool channel member 170 that carries a set of tool channels 130.

More particularly, the secondary probe member 270 can be independently or separately controlled relative to the tool channel member 170 such that the secondary probe member 270 can be selectively positioned relative to the primary endoscope probe's central axis, the tool channel member 170, and a central axis of each tool channel 130.

In an embodiment, the tool channel member 170 can be a distal extension of a cross sectional portion of the primary endoscope probe body 110 that carries the set of tool channels 130. The secondary probe member 270 can be a distal extension of a cross sectional portion of the primary endoscope body 110 that carries the secondary endoscope probe channel 240. The secondary probe member 270 has a distal end 274 and the tool channel member 170 has a distal end 174, where each such distal end 274, 174 can define or terminate at the primary endoscope probe body's distal end 140. That is, in a number of embodiments, the secondary probe member 270 and the tool channel member 170 share a common termination point or plane, or have an identical, essentially identical, or substantially identical length.

For purpose of simplicity and to aid understanding, in non-limiting representative embodiments described hereafter, the secondary probe member 270 includes or is primarily intended to provide endoscopic imaging functionality. In the embodiment shown in FIGS. 4A and 4B, an imaging member 270 includes a camera module 222, a number of illumination sources 224, and possibly an adjunct endoscopy element (e.g., an insufflation aperture) 226 at its distal end 274.

The imaging member 270 also includes structural elements therein which facilitate or enable the selective (a) position locking of the imaging member 270 directly adjacent to, upon, or against the tool channel member 170; and (b) positioning of portions of the imaging member 270 away from or above the tool channel member 170. For instance, the imaging member 270 can include cable elements and vertebrae-type joint elements configured for counterflex motion of proximal and distal portions of the secondary probe member 270, in a manner analogous or generally analogous to that described above with respect to the S-bend imaging endoscope 200 shown in FIGS. 3A-3D. Thus, a proximal controllable region 280a of the secondary probe member 270 which is configured for providing heave displacement can selectively elevate the imaging member's distal end 274 away from and above the primary endoscope probe's central axis (and hence above each tool channel's central axis); and a distal controllable region 280b of the imaging member 270 which is configured for counterflexion relative to the proximal controllable region 280a can position the camera module 222 such that its field of view is selectively oriented toward the primary endoscope probe's central axis within a spatial region beyond the distal end 104 of the primary endoscope probe 100 within which a set of robot arms 400 and corresponding end effectors can operate. The camera module 222 can correspondingly selectively capture anterograde and retrograde images relative to a set of target sites at which the robot arm(s) and end effector(s) can be positioned or interact with target tissue(s). In certain embodiments, the imaging member 270 can additionally or alternatively include structural elements configured for providing selective sway displacement of the imaging member 270.

FIGS. 5A and 5B are schematic illustrations of a primary endoscope probe 100 that includes a secondary probe member 270 in accordance with another embodiment of the present disclosure. In an embodiment, each of the secondary probe member 270 and the tool channel member 170 have an outer or exterior surface that, when the secondary probe member 270 rests upon/adjacent to, is disposed substantially flush with, or is position locked against the tool channel member 170, uniformly maintain or substantially uniformly maintain the outer or exterior shape or profile of the primary endoscope probe body 110 from the primary endoscope probe's proximal end 102 to its distal end 104. The secondary probe member 270 is selectively separable from and positionable relative to the tool channel member 170, in a manner analogous to that described above with reference to FIGS. 4A and 4B.

In view of the foregoing, depending upon embodiment details a primary endoscope probe 100 can be configured for carrying various types of secondary endoscope probes 200 or probe modules 270, such as imaging endoscopes 200 or imaging members 270 that carry camera modules 222, and which are configured/configurable for selectively/selectably positioning such camera modules 222 to capture anterograde and/or retrograde images of a target site at which one or more robot arms 400 and corresponding effectors are disposed, within a spatial volume in which the robot arm(s) and effector(s) are manipulable or positionable.

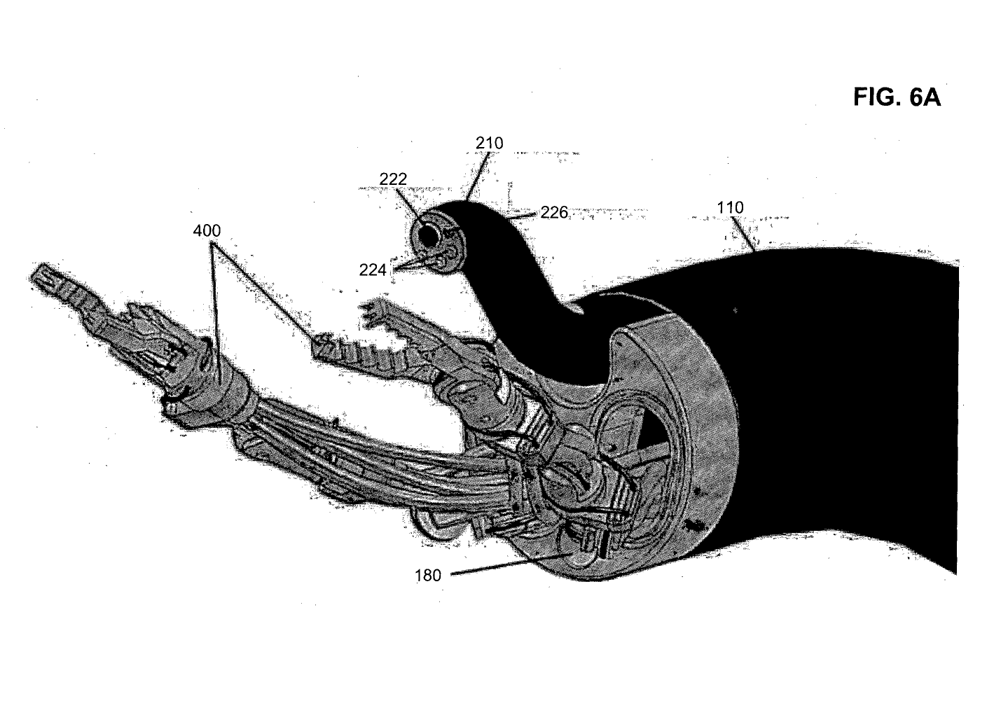

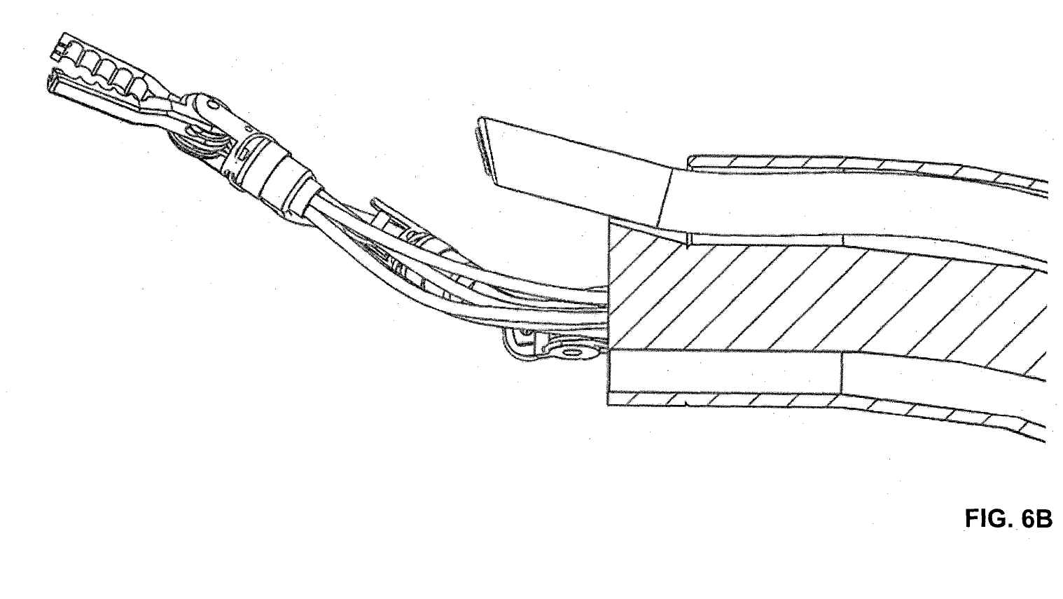

For instance, FIG. 6A is a perspective view of a representative embodiment of a primary endoscope probe 100 corresponding to that described above with reference to FIG. 3A, which is configured for carrying a first robot arm 400a, a second robot arm 400b, and an S-bend imaging endoscope 200 having a camera module 222, a set of LEDs 224, and an insufflation aperture 226. Similarly, FIG. 6B is a perspective view of a representative embodiment of a primary endoscope probe 100 corresponding to FIG. 3F, which is configured for carrying a first robot arm 400, a second robot arm 400, and a bevel tip imaging endoscope 200 in accordance with an embodiment of the present disclosure. Each of the S-bend imaging endoscope 200 and the bevel tip imaging endoscope 200 is configured for surge displacement as well as heave displacement, and can additionally be configured for sway displacement to facilitate or enable the capture of anterograde and/or retrograde images.

In addition to the foregoing, in some embodiments the secondary endoscope probe 200 is configured for selective, adjustable, or controllable rotation or roll motion about its central/longitudinal axis Zₛ (or analogously/correspondingly, about the primary endoscope probe central axis Zₚ). For instance, an imaging endoscope 200 such as described above with reference to FIGS. 3A-3K can be configured for automated and adjustable/controllable (a) surge displacement along the primary endoscope probe central axis Zₚ; (b) heave displacement relative to the primary endoscope probe central axis Zₚ; (c) sway displacement relative to the primary endoscope probe central axis Zₚ; (d) rotation or roll about its own central/longitudinal axis Zₛ; and/or another type of motion, such as yaw motion about its own vertical axis Y. Depending upon embodiment details, secondary endoscope probe positioning or manipulation can be adjusted or controlled by way of the endoscopist interface 30, and/or the master console 1000 (e.g., on a selective basis). In embodiments in which the secondary endoscope probe 200 is configured for such rotation or roll motion, the proximal portion of the primary endoscope probe 100, the endoscopist interface 30, or the translation mechanism 40 can carry an actuation element configured for receiving/carrying a portion of the secondary endoscope probe and selectively and controllably rotating the secondary endoscope probe 200, in a manner understood by one having ordinary skill in the relevant art. In certain embodiments, a secondary endoscope probe member 270 can be configured for at least some amount of rotation about the primary endoscope probe central axis Zₚ, such as by way of the inclusion of a rotational joint primitive (described in detail below) within a portion of the secondary endoscope probe member 270. Embodiments in which a portion of the secondary endoscope 200 or the secondary endoscope probe member 270 are configured for providing yaw motion can include a rotational joint primitive to facilitate or enable such motion.

In various embodiments the first and second robot arms 400a,b, as well as tendon-sheath elements 330 and tendons 334 corresponding thereto, are carried by disposable actuation assemblies 300 configured for removable insertion into the primary endoscope probe's tool channels 130a,b. Aspects of representative disposable actuation assemblies 300 and robot arms 400 in accordance with embodiments of the present disclosure are described in detail hereafter.

Aspects of Disposable Actuation Assembly Embodiments

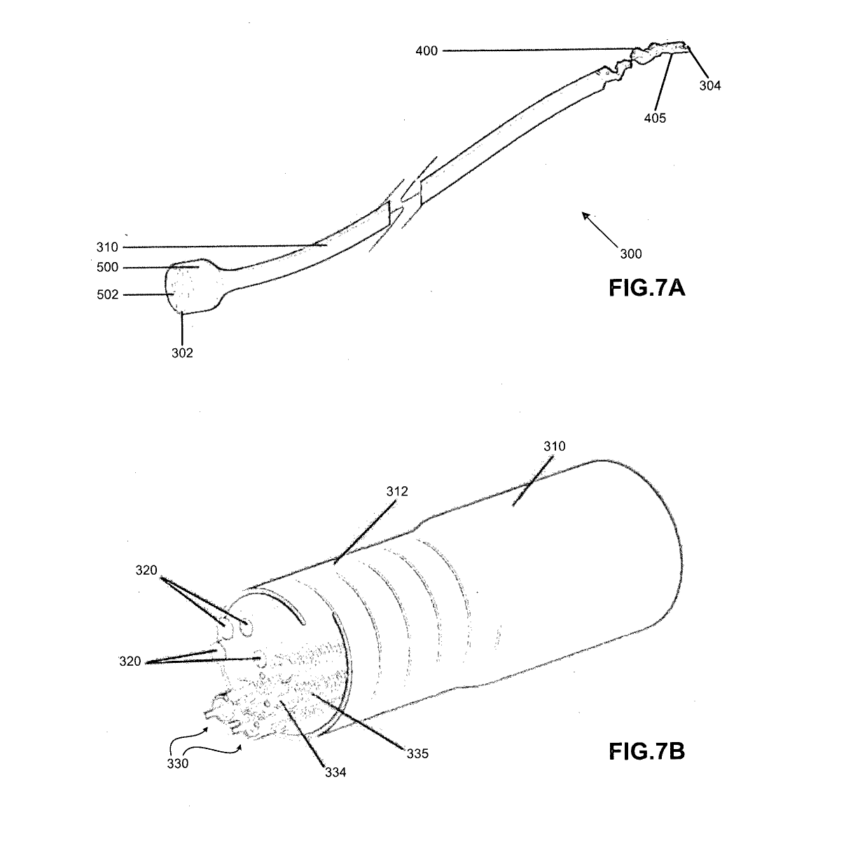

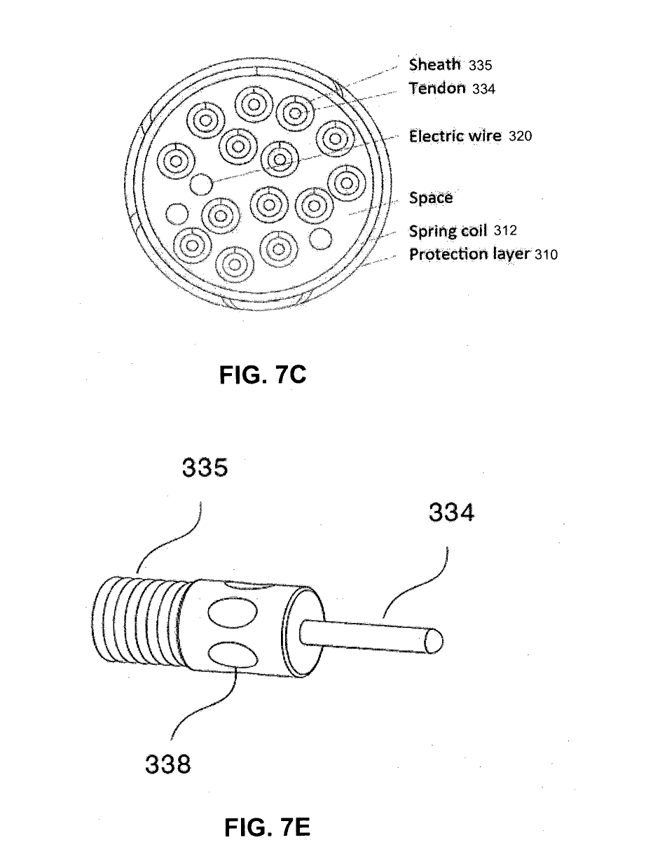

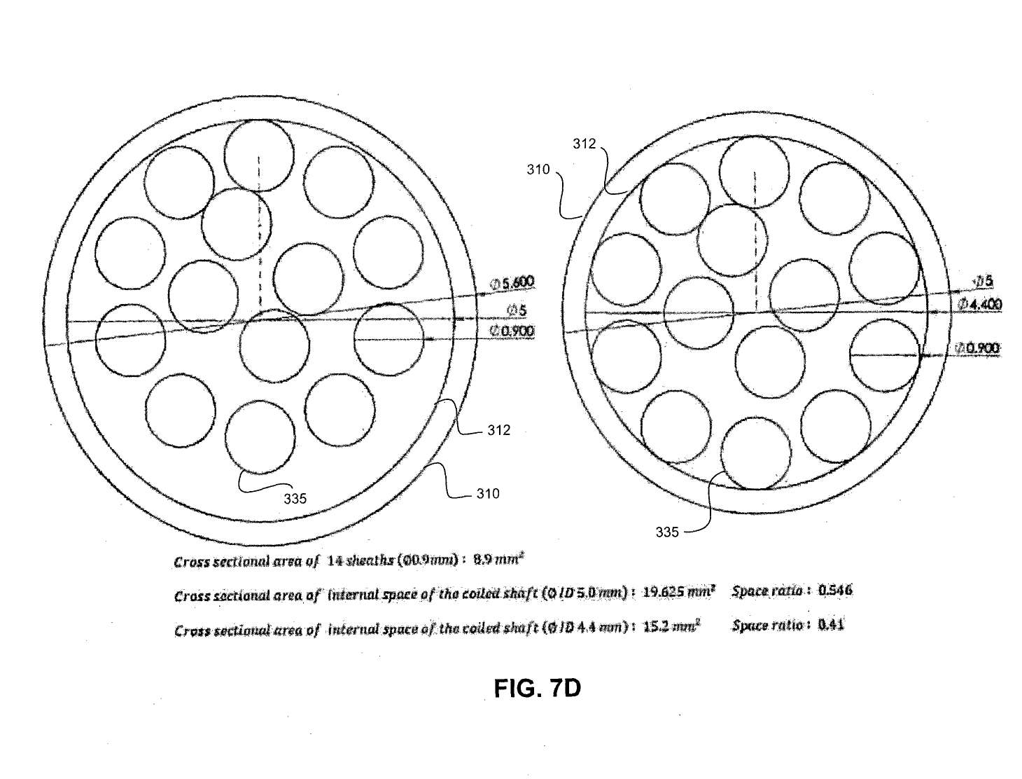

FIG. 7A is a schematic illustration of a flexible or substantially flexible disposable actuation assembly 300 in accordance with an embodiment of the present disclosure. In an embodiment, the disposable actuation assembly 300 includes a body or outer sleeve 310 which is configured for carrying tendon-sheath and/or other types of elements (e.g., electromagnetic signal elements) therein. The disposable actuation assembly 300 also includes a distally carried or supported robot arm 400, to which an effector or end effector 405 can be coupled; and a proximally carried quick release interface 500 that facilitates releasable coupling of the disposable actuation assembly 300 to the actuation controller 700. An engagement surface 502 of the quick release interface 500 can define a proximal end 302 of the disposable actuation assembly 300, and a most-distal portion or tip of an effector 405 can define a distal end 304 of the disposable actuation assembly 300.

With additional reference to FIG. 1B, the quick release interface 500 can establish or define a boundary or border between endoscope-side elements of the system 10 and actuator-side elements of the system 10, where the disposable arm assembly 300, the endoscopist interface 30, and the primary endoscope probe 100 correspond to endoscope-side system elements, and the actuation controller 700 and its control unit 800 correspond to actuator-side system elements. As further detailed below, matingly engagable endoscope-side and counterpart actuator-side quick connect/disconnect interfaces can be configured for providing an environmental barrier, such as a pathogen controlled or sterile barrier, between endoscope-side and actuator-side system elements.

The disposable actuation assembly's outer sleeve 310, the robot arm 400, and the effector 405 have a maximum cross sectional area or diameter that is intended to coordinate with the cross sectional areas of (a) ports or openings provided by the endoscopist interface 30; and (b) a set of tool channels 130 provided by the primary endoscope probe 100. Furthermore, the disposable actuation assembly 300 has an overall length that is greater than the length of the primary endoscope probe 100. Consequently, the effector 405, the robot arm 400, and a substantial length of the outer sleeve 310 can be inserted into a port provided by the endoscopist interface 30, and fed into and through the primary endoscope probe 100 until the robot arm 400 and effector 405 extend beyond the distal end 104 of the primary endoscope probe 100.

Once the robot arm 400 and effector 405 protrude from the distal end 104 of the primary endoscope probe 100 and are disposed in an appropriate deployment configuration relative to the distal end 104 of the primary endoscope probe 100, and are retained, secured, or locked in the deployment configuration, portions of the outer sleeve 310 extend away from and remain external to the endoscopist interface 30. The disposable actuation assembly's quick release interface 500 can be coupled to a counterpart actuation-side quick release interface to facilitate or enable the transfer of electromagnetic signals and/or mechanical forces between the actuation controller 700 and the disposable actuation assembly 300. As indicated above, a primary endoscope probe tool channel 130 into and along which portions of the disposable actuation assembly 300 is insertable can include a docking mechanism (e.g., a brace element) disposed near or at the tool channel's distal end, such that the robot arm 400 and effector 405 can be securely, yet releasably, maintained in the deployment configuration. In a number of embodiments, the disposable actuation assembly 300 can include one or more docking features (e.g., a collar, and/or protruding or recessed structural elements) carried by its outer sleeve 310 and/or a base portion of the robot arm 400 to facilitate such docking relative to the primary endoscope probe 100.

FIG. 7B is a perspective schematic illustration and FIG. 7C is a cross sectional schematic illustration a flexible or substantially flexible disposable actuation assembly 300 in accordance with an embodiment of the present disclosure. In an embodiment, the disposable actuation assembly 300 includes a flexible or substantially flexible helical spring 312 internal to its outer sleeve 310, which carries one or both of a set of flexible or substantially flexible electromagnetic signal transfer lines 320 (e.g., wires for carrying electrical signals, and/or optical fibers for carrying optical signals) and a set of flexible or substantially flexible tendon-sheath elements 330. The helical spring 312 can support and protect the elements surrounded thereby. The outer sleeve 310 can include a biocompatible layer or coating, such as a biocompatible polymer or epoxy layer/coating, which surrounds the helical spring 312.

A tendon-sheath structure 330 includes a flexible or substantially flexible cable or tendon 334 that is surrounded by a corresponding flexible or substantially flexible sheath 335, such as a hollow helical coil. The tendon-sheath structure 330 is configured for providing slidable lengthwise or longitudinal displacement of the tendon 334 within the sheath 335 in response to forces (e.g., pulling forces) applied to the tendon 334 (e.g., forces generated by the actuation controller 700 and communicated to the tendon 334 by way of the quick release interface 500). Such longitudinal tendon displacement can transfer or transmit the forces applied to the tendon 334 to a joint element or articulation structure to which the tendon 334 is coupled, thereby facilitating manipulation of the joint element in an intended manner (e.g., corresponding to positioning a robot arm 400 and/or end effector 405).

The number of electromagnetic signal transfer lines 320 and tendon-sheath structures 300 carried by a disposable actuation assembly 300 depends upon a type of robot arm 400 and/or effector 405 under consideration. More particularly, the number of tendon-sheath structure 300 depends upon the DOF requirements associated with the robot arm 400 and effector 405 (which correspondingly depends upon a type of surgical intervention under consideration). Different types of effectors 405 (e.g., graspers, scissors, cautery hooks, blades, etc . . . ) can exhibit different intended DOFs. An effector 405 is typically the distal-most or final portion of the disposable actuation assembly 300, and can be defined as the “last link” of a robot arm 400. Therefore, a set of additional DOFs are needed for the robot arm 400, in accordance with which the robot arm 400 can appropriately position or orient the effector 405 such that the effector 405 can carry out its intended functionality.

In various embodiments, each DOF is provided by way of two tendons 334, and hence two tendon-sheath structures 320 are utilized for each DOF in accordance with which the robot arm 400 can be manipulated. Thus, if a particular robot arm 400 has N DOFs, a disposable actuation assembly 300 corresponding to this robot arm 400 includes 2N tendon-sheath structures 330, which can mechanically couple portions of the robot arm 400 to the actuation controller 700 by way of the quick release interface 500.

If tendon-sheath structures 330 are packed too densely within a disposable actuation assembly 300, the flexibility of the disposable actuation assembly 300 can be reduced or compromised. In order to provide and maintain flexibility, substantial, or maximum flexibility, the interior of a disposable actuation assembly 300 should include or provide for a certain amount of reserve space or reserve spatial volume beyond the space or spatial volume occupied by the tendon-sheath structures 330 carried thereby.

FIG. 7D is a cross sectional schematic illustration of a representative relationship between the internal space or cross sectional area provided by a disposable actuation assembly 300 and the overall internal space or cross sectional area within the disposable actuation assembly 300 occupied by its tendon-sheath structures 330, which can facilitate the provision and/or maintenance of significant or substantial disposable actuation assembly flexibility. In the embodiment shown in FIG. 7D, the disposable actuation assembly 300 is configured for carrying up to fourteen tendon-sheath structures 300 while remaining flexible or substantially flexible essentially or substantially regardless of the manner in which the primary endoscope probe distal end 104 has been navigated into a target environment.

In multiple embodiments, at least some tendon-sheath structures 330 include a termination element. FIG. 7E is a schematic illustration of a tendon-sheath structure 330 having a sheath termination element 338 in accordance with an embodiment of the present disclosure. In an embodiment, the sheath termination element 338 can include a cap, which can be overmolded or crimped onto a terminal portion, section, or end of a sheath 335.

Aspects of Representative Joint Primitives and Robot Arms Any given robot arm 400 is configured for positioning or moving an effector 405 carried thereby to facilitate effector positioning and/or interaction with respect to a target anatomical environment, region, or tissue. A robot arm 400 in accordance with embodiments of the present disclosure can include one or more types of joint elements, which can include particular types of fundamental, basis, or primitive joint structures that can facilitate the enhancement or maximization of the (a) payload that the robot arm 400 can reliably, carry or handle, and/or (b) forces that the robot arm 400 and its effector 405 can reliably apply or withstand. Such fundamental joint structures can be used singly or in combination for providing the robot arm 400 with desired or intended DOFs by way of tendon-sheath based transmission and application of mechanical forces.

Vertebra Joint Primitives

FIG. 8A is a schematic illustration of a representative vertebra joint primitive 410 in accordance with an embodiment of the present disclosure. In an embodiment, the vertebra joint primitive 410 includes a proximal body portion 420 and a distal body portion 422, each of which includes an outer periphery. The proximal body portion 420 has a cross sectional area, and a central or longitudinal proximal body portion axis can be defined perpendicular to this cross sectional area, extending through a proximal body portion center or centroid. Similarly, the distal body portion 422 has a cross sectional area, relative to which a central or longitudinal distal body portion axis can be defined that extends through a distal body portion center or centroid. In several embodiments, each body portion's cross sectional area is circular or approximately circular; however, in other embodiments, a body portion's cross sectional area can correspond to another geometric shape. An exposed portion (e.g., a rim or lip) of the proximal body portion 412 transverse to the central axis of the proximal body portion 420 can define a proximal end 412 of the vertebra joint primitive 410; and an exposed portion (e.g., a rim or lip) of the distal body portion 422 transverse to the central axis of the distal body portion 422 can define a distal end 414 of the vertebra joint primitive 410.

The proximal body portion 420 is configured for carrying the distal body portion 422 by way of pivotable mating engagement, which can involve counterpart protrusion/recess structures. For instance, in the embodiment shown in FIG. 8A, the proximal body portion 420 includes a pair of recesses 418 carried thereby (e.g., integrally formed therein), and the distal body portion 422 includes a pair of protrusions 428 carried thereby (e.g., integrally formed therein), where each recess 418 is configured for receiving and securely retaining a portion of a protrusion 428 in a manner that enables pivotable displacement of the protrusion 428 within the recess 418. A protrusion-recess pair can be a disc-in-cup structure, in a manner that will be understood by one of ordinary skill in the relevant art.

When the central or longitudinal axes of the proximal and distal body portions 420, 422 are aligned (i.e., in the absence of pivotal displacement of the distal body portion 422 relative to the proximal body portion 420) they define or coincide with a central or longitudinal axis of the vertebra joint primitive 410.

The proximal body portion 420 includes at least two tendon channels or guides 430 carried by opposite internal sides of the proximal body portion 420; and the distal body portion 422 includes at least two corresponding tendon coupling structures 434 carried by on opposite internal sides of the distal body portion 422. In the absence of pivotal displacement of the distal body portion 422 relative to the proximal body portion 420, a given proximal tendon guide 430 is axially or longitudinally aligned with a corresponding distal tendon coupling structure 434. A tendon guide 430 is configured for providing a channel through which a tendon 334 can slidably pass, and a tendon coupling structure 434 is configured for receiving and securely coupling or linking to the tendon 334 that passes through its counterpart tendon guide 430.

When tension is differentially applied (e.g., by way of pulling forces generated by the actuation controller 700) to tendons 334 carried on opposite internal sides of the joint primitive element 410, an increase in the tension applied to one tendon 334 relative to the other tendon 334 causes the distal body portion 422 to pivot relative to the proximal body portion 420. Such pivot displacement causes the joint primitive element 410 to flex in accordance with either yaw or pitch motion, in a manner that will be understood by one of ordinary skill in the relevant art.

In various embodiments, a vertebra joint primitive's proximal and distal body portions 420, 422 have a substantially hollow cross section, into or through which tendon-sheath elements 330 or tendons 334 can extend. As a result, tendon-sheath elements 330 or tendons 334 disposed within the vertebra joint primitive's hollow cross section are protected from the vertebra joint primitive's external environment, which can reduce wear or abrasion thereupon.

Rotational Joint Primitives

FIG. 8B is a schematic illustration of a representative rotational joint primitive 440 in accordance with an embodiment of the present disclosure. In an embodiment, the rotational joint primitive 440 includes a drum member 442 having an outer periphery and a cross sectional area, transverse or perpendicular to which an axis of rotation can be defined, which extends through a center or centroid of the drum member 442. The drum member 442 is configured for securely carrying and retaining portions of a tendon 334 wrapped thereabout. A tension or pulling force differentially applied to a first end of the tendon 334 relative to a second end of the tendon 334 causes rotation of the drum member 442. For instance, if the first end of the tendon 334 is exposed to a given pulling force while the second end of the tendon 334 is exposed to a smaller or zero pulling force, the drum member 442 can rotate in a first direction (e.g., clockwise). Similarly, if the second end of the tendon 334 is exposed to a given pulling force while the first end of the tendon 334 is exposed to a smaller or zero pulling force, the drum member can rotate in a second direction (e.g., counterclockwise).

A rotational joint primitive 440 can be disposed or inserted proximal or distal to, or in between the above described vertebra joint primitives 410. Such rotational joint primitives 440 facilitate or enable robot arm rotation about the rotational joint primitive's axis of rotation, which can correspond to or coincide with the robot arm's central or longitudinal axis. By way of selective coordination or combination of vertebra joint primitives 410 and rotational joint primitives 440, a robot arm 400 can provide or support a desired or intended number of DOF.

Representative Combination of Vertebra and Rotational Joint Primitives in a Robot Arm

FIGS. 8C-8E are a schematic side view, cross sectional view, and top view, respectively, of a robot arm 400 that includes vertebra joint primitives 410 and rotational joint primitives 440, and which is configured for selective motion in six DOFs, in accordance with an embodiment of the present disclosure. As indicated in FIGS. 8C-8E, vertebra joint primitives 410 and rotational joint primitives 440 can be selectively disposed in sequence or stacked to define multi-segment robot arms 400, where any given segment is associated with a DOF provided by its vertebra or rotational joint primitive 410, 440.

Representative Revolute Joint Primitives

Another categorical type of joint primitive is based upon the coupling or linking of a set of tendons 334 to a revolvable body such as a pulley, and the selective application of forces (e.g., pulling forces) to the set of tendons 334 to effect revolution of the pulley in an intended direction. In multiple embodiments, the selective revolution of a given pulley is controlled/controllable by way of a pair of tendons 334 coupled, linked, or secured to the pulley.

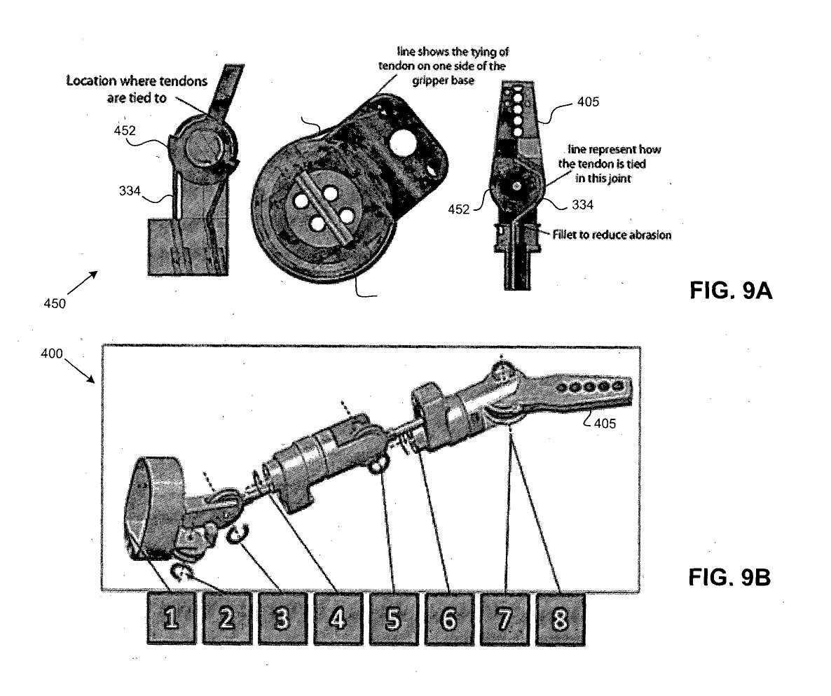

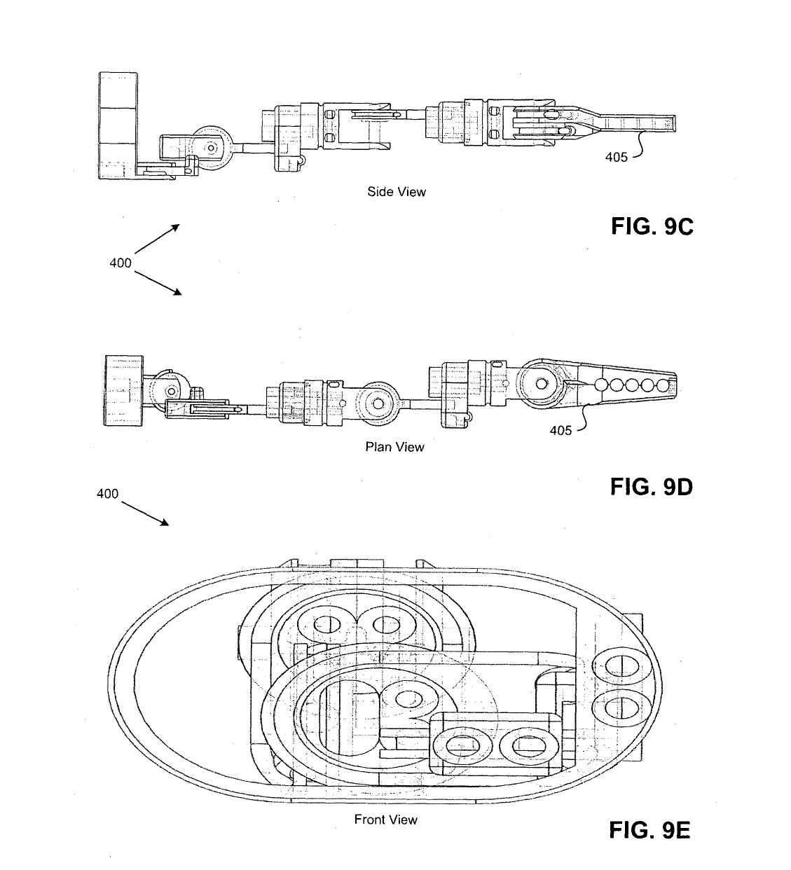

FIG. 9A is a schematic illustration of representative revolute joint primitives 450 in accordance with an embodiment of the present disclosure. As indicated in FIG. 9A, tendons 334 can be secured to a revolute element such as a pulley 452 in a variety of manners, such that a pulling force applied to a given tendon 334 causes the pulley 452 to which the tendon 334 is linked or attached to revolve or rotate in a given direction about a pulley axis of rotation. The pulley axis of rotation extends through a pulley center or centroid, and is perpendicular to a pulley cross sectional area or diameter. The pulley axis of rotation can equivalently be defined as a revolute joint axis of rotation. In various embodiments, a pulling force applied to a first tendon 334 can cause the pulley 452 to rotate in a first direction, and a pulling force applied to a second tendon 334 can cause the pulley 452 to rotate in a second direction opposite to the first direction.

A revolute joint primitive 450 can be incorporated into a robot arm 400 in a manner that establishes an intended or desired revolute joint axis of rotation with respect to a central axis of the robot arm 400. As a result, the robot arm 400 is provided with a revolute or rotational DOF about the revolute joint primitive 450. Analogously, multiple revolute joint primitives 450 can be carried by or along different portions or segments of a robot arm 400 in order to provide the robot arm 400 with selective manipulability through an intended or desired number of revolute DOFs.

Representative Combination of Revolute Joint Primitives in a Robot Arm

FIG. 9B is a schematic illustration of a robot arm 400 which includes multiple revolute joint primitives 450, and which is configured for selective motion in eight DOFs in accordance with an embodiment of the present disclosure. A first DOF can be controlled by way of translating the entire robot arm 400 proximally or distally relative to the primary endoscope probe's distal end 104, by way of the translation mechanism 40. A second through an eighth DOF can be controlled by way of revolute joint primitives 450 disposed at predetermined portions of the robot arm 400, and having their revolute joint axes of rotations in predetermined orientations relative to the central axis of the robot arm 400 to support each desired or intended DOF. In the embodiment shown, the second through eighth DOF can correspond to shoulder medial rotation; elbow flexion/ extension; forearm supination/pronation; wrist flexion/extension; and first and second finger opposition/reopposition (i.e., grasping), in a manner that will be understood by one of ordinary skill in the relevant art. FIGS. 9C-9E are side, plan, and front orthogonal projection views of the robot arm 400 of FIG. 9B. One of ordinary skill in the relevant art will recognize that the robot arm embodiment shown in FIGS. 9B-9E corresponds to the robot arms 400 shown in FIG. 6.

In addition or as an alternative to the foregoing, a robot arm 400 can include multiple different/distinct types of joint primitives, for instance, two or more of vertebra joint primitives 410, rotational joint primitives 440, and revolute joint primitives 450 in accordance with embodiments of the present disclosure. Such distinct types of joint primitives can be selectively disposed along particular portions of a robot arm 400 (e.g., disposed in sequence, or stacked relative to robot arm segments) in order to provide the robot arm 400 with manipulability through intended DOFs.

Aspects of a Representative Endoscopist Interface

With reference again to FIG. 1B, in an embodiment the endoscope-side of the system 10 includes a primary endoscope 20 having a primary endoscope probe 100 that is configured for carrying each of a secondary endoscope probe or probe module 200 (e.g., an imaging endoscope) and at least one disposable actuation assembly 300 that includes or supports a robot arm 400 and its effector 405. The primary endoscope 20 additionally includes an endoscopist interface 30 from which the primary endoscope probe 100 extends.

The endoscopist interface 30 includes a set of ports or openings that facilitates or enables (a) the insertion of disposable actuation assemblies 300 into and along the length of the primary endoscope probe 100, such that robot arm(s) 400 and effector(s) 405 can extend beyond the distal end 104 of the primary endoscope probe 100; and (b) the selective longitudinal translation of the disposable actuation assemblies 300 in a direction parallel to or along the primary endoscope probe's central axis (e.g., by way of the translation mechanism 40) after the disposable actuation assemblies 300 have been secured in a deployment position within the primary endoscope probe 100, such that the robot arm(s) 400 and effector(s) 405 can correspondingly be selectively longitudinally translated within or across a spatial volume external to the primary endoscope probe's distal end 104. Further aspects of a representative embodiment of an endoscopist interface 30 are described hereafter.

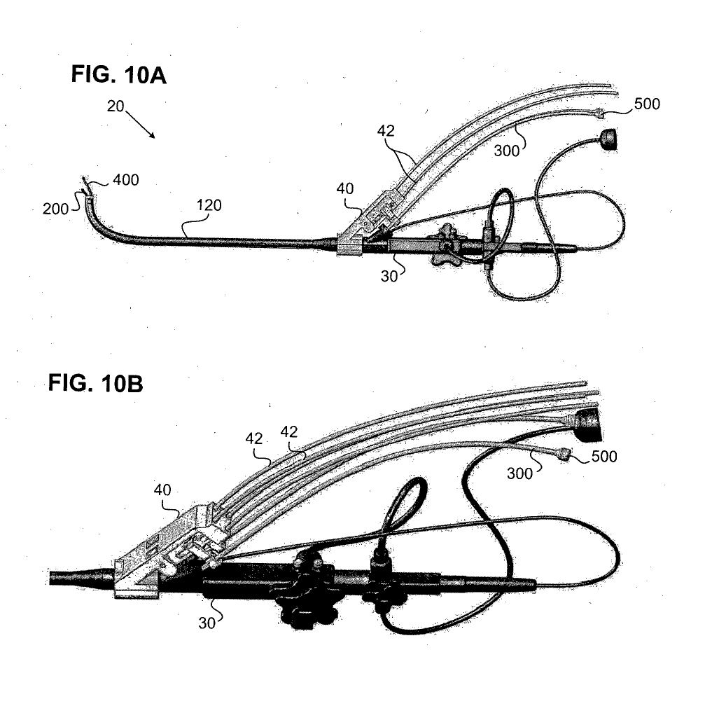

FIGS. 10A and 10B are schematic illustrations of an endoscopist interface 30 and a translation mechanism 40 in accordance with an embodiment of the present disclosure. In an embodiment, the endoscopist interface 30 and the translation mechanism 40 are configured for carrying the disposable actuation assemblies 300. The translation mechanism 40 includes a number of actuators (e.g., linear actuators) configured for selectively axially translating or displacing one or more disposable actuation assemblies 300. Such actuators can be coupled or linked to axial translation links 42, which can be coupled to the actuation controller 700, for instance, by way of quick release structures or interfaces that are substantially identical or similar to the quick release interfaces 500, 600 by which disposable actuation assemblies 300 can be selectively coupled to and decoupled from the actuation controller 700.

Aspects of Representative Quick Release Connectors

A set of quick release interfaces 500, 600 in accordance with an embodiment of the present disclosure facilitates detachable coupling or attachment between disposable actuation assemblies 300 that can carry various types of surgical instruments and the actuation controller 700. In various embodiments, quick release interfaces 500, 600 facilitate or effectuate the transfer of rotational mechanical energy to linear motion of endoscope-side paired tensile tendons or tendon sections/segments 334 within their corresponding sheaths 335.

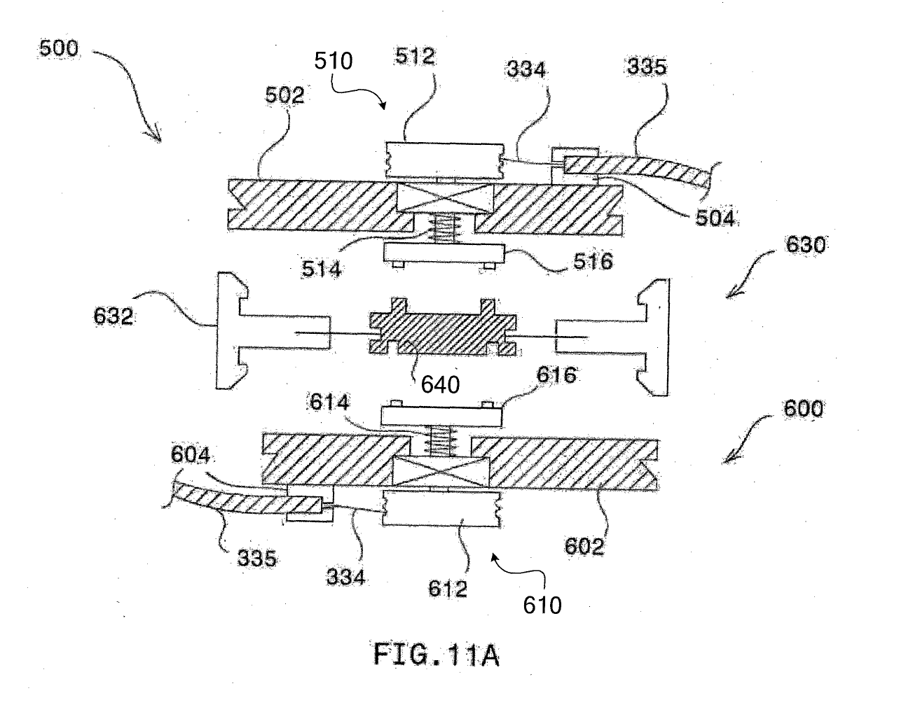

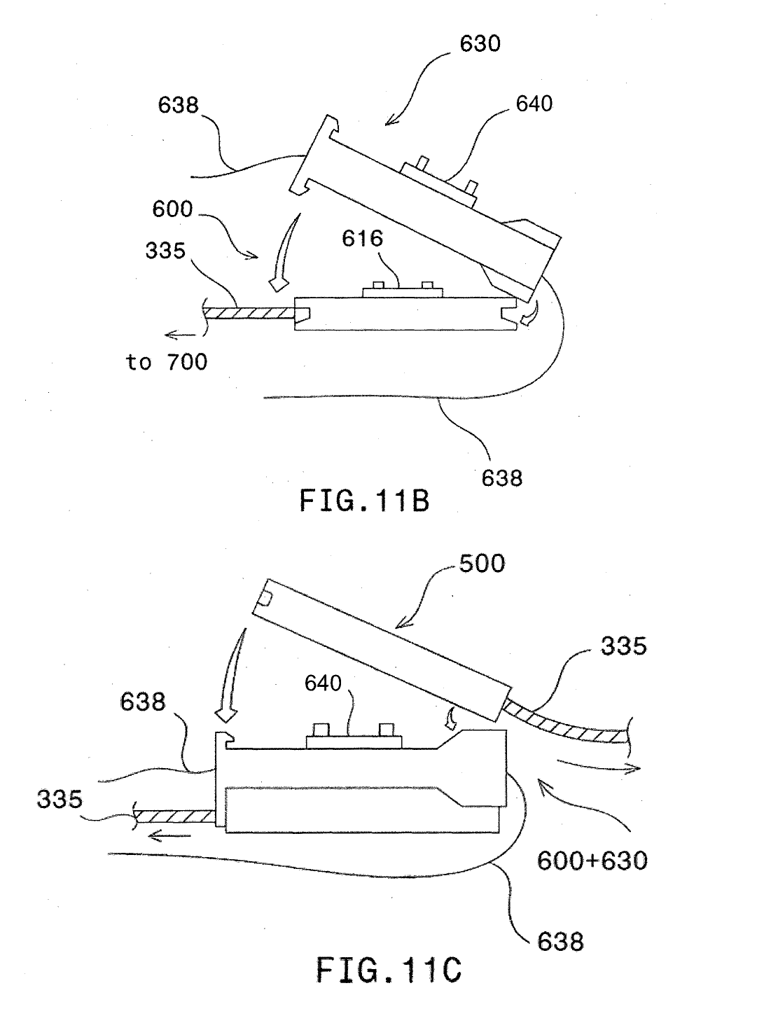

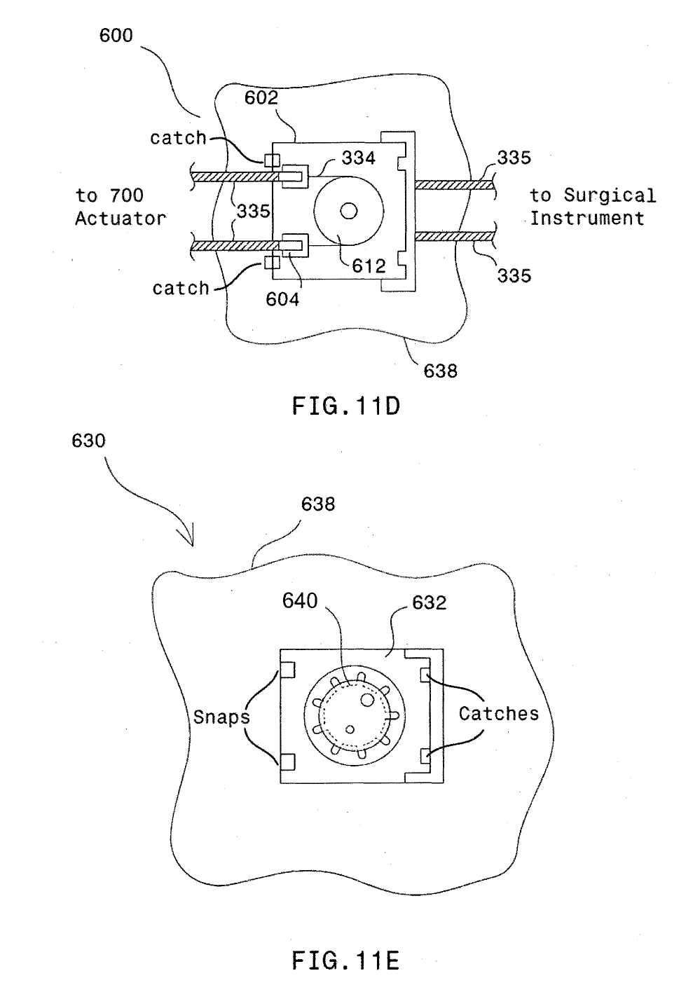

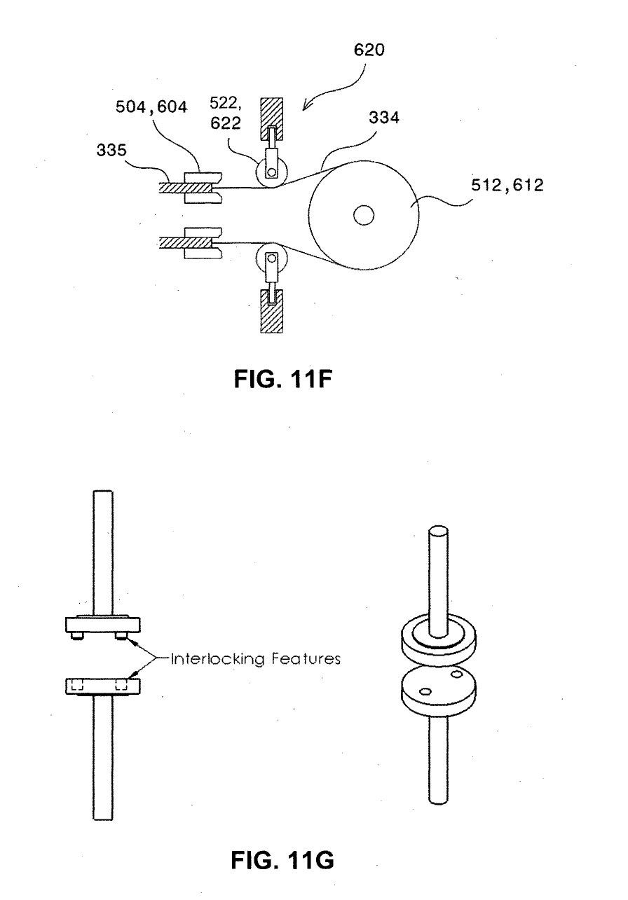

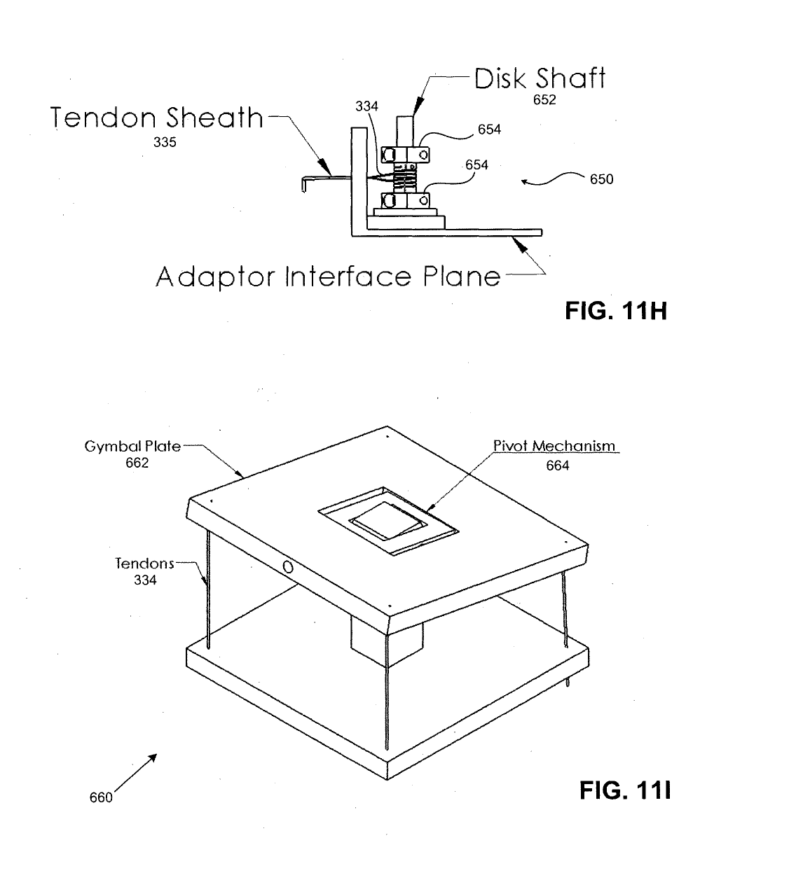

FIGS. 11A-11E are schematic illustrations of quick release interfaces 500, 600, 630 that are coupled/couplable to form a quick release assembly in accordance with an embodiment of the present disclosure. In an embodiment, a quick release assembly includes an actuator-side quick release interface 600 that can be mechanically coupled to an endoscope-side quick release interface 500 such that linear motion of an actuator-side tendon 334 results in linear motion of an endoscope-side tendon 334.

The endoscope-side and actuator-side quick release interfaces 600, 500 are configured for (a) mating snap-fit detachable engagement with each other, as well as (b) mating tendon mechanical energy transfer between each other. In specific embodiments, the endoscope-side quick release interface 500 and the actuator-side quick release interface 600 can be configured for direct mating snap-fit engagement with each other. However, in multiple embodiments described below, the endoscope-side quick release interface 500 and the actuator-side quick release interface 600 are structurally coupled by way of an intermediary quick release interface 630, which can carry or be attached to portions of an environmental barrier as described hereafter.

An intermediary quick-release barrier interface 630 can be configured for providing a mechanical energy pass-through, and can be further configured for carrying or providing an environmental barrier 638 such as a surgical/sterile drape that facilitates environmental segregation or isolation between actuator-side elements of the system 10 and endoscope-side elements of the system 10. As indicated in FIGS. 11B-11E, an environmental barrier 638 can be configured to cover or isolate the actuator-side quick release interface 600, the actuation controller 700, and couplings therebetween from endoscope-side system elements.

The actuator-side quick release interface 600 includes a housing 600 that carries a sheath support element 604 configured for receiving and supporting an actuator-side sheath 335 that carries an actuator-side tendon 334. The actuator-side quick release interface 600 also carries an actuator-side mechanical energy/motion/force delivery structure 610. In a similar or analogous manner, the endoscope-side quick release interface 500 includes a housing 502 that carries a sheath support element 504 configured for receiving and supporting an endoscope-side sheath 335 that carries an endoscope-side tendon 334, which extends through a disposable actuation assembly 300 and is coupled to a robot arm 400. The endoscope-side quick release interface 500 also carries an endoscope-side mechanical energy/motion/force receiving structure 510. The intermediary quick release barrier interface 630 includes a housing 632 that carries an intermediary mechanical energy/motion/force communication, transfer, bridging, or linkage structure 640. By way of the actuator-side force delivery structure 610, the intermediary force bridging structure 640, and the endoscope-side force receiving structure 510, linear motion of or linear force applied to an actuator-side tendon 334 is converted to rotational motion by the quick release interfaces 500, 600, 630, and converted to linear motion of or linear force applied to the endoscope-side tendon 334.

In some embodiments, tendon linear motion or force is converted to rotational motion by way of a wheel or pulley element, structure, or device to which a tendon 334 is coupled, linked, or wrapped relative to or about a portion of a pulley's circumference, for instance, in a manner indicated in FIG. 11D. In a number of embodiments, tendon slack or stretch, which can be induced or introduced by longitudinal mechanical stress upon the tendon 334 (e.g., over time), can be accommodated by a tendon tensioning mechanism 520, 620 such as that shown in FIG. 11F, which includes a spring-loaded pulley 522, 622 configured to apply a lateral force to a tendon 334 in a direction transverse or perpendicular to the tendon's length. One or more tendon tensioning mechanisms 520, 520 can be carried by one or each of the endoscope-side quick release interface 500 and the actuator-side quick release interface 600.

The actuator-side force delivery structure 610 can include a pulley 612 having a circumference relative to which an actuator-side tendon 334 is wrapped. The actuator-side pulley 612 is coupled to a rotatable shaft 614, which can further be coupled to a rotatable mating engagement disk 616. This rotatable shaft 614 and this disk 616 can also be considered as portions of the actuator-side force delivery structure 610. Similarly, the endoscope-side force receiving structure 510 can include a pulley 512 having a circumference relative to which an endoscope-side tendon 334 is wrapped, where the endoscope-side pulley 512 is coupled to a rotatable shaft 514, which can be further coupled to a rotatable mating engagement disk 516. This rotatable shaft 514 and this disk 516 can also be considered as portions of the endoscope-side force receiving structure 510.

The intermediary force bridging structure 640 includes or is a rotatable force communication disk that is matingly engageable with each of (a) the actuator-side force delivery structure mating engagement disk 616, and (b) the endoscope-side force delivery structure mating engagement disk 516, and serves as a mechanical energy pass-through structure. Such mating engagement can occur by way of locking structures such as corresponding or counterpart protrusions, apertures, recesses, etc . . . carried by the force communication disk, the actuator-side quick release interface mating engagement disk 616, and the endoscope-side quick release interface mating engagement disk 516, for instance, in a manner indicated in FIG. 11G and readily understandable by one of ordinary skill in the relevant art. The rotatable force communication disk is carried or suspended by the intermediary force bridging interface housing 642 in a manner that facilitates smooth, low or minimal friction transfer of rotational mechanical energy. In some embodiments, the intermediary force bridging interface 630 includes a suspension structure such as a spring-loaded finger suspension and/or a set of bearing elements, such as thin-section precision section-ball or ring-type bearings that facilitate or enable such smooth, low/minimal friction rotational energy transfer, in a manner understood by one of ordinary skill in the relevant art.

Rotation of the actuator-side quick release interface pulley 612 in response to linear motion of or linear force applied to the actuator-side tendon 334 (e.g., one side of the actuator-side tendon 334 versus the other side of the actuator-side tendon 334, with respect to the pulley's circumference, depending upon the direction of rotation of the pulley 612) results in rotation of the actuator-side quick release shaft 614 and mating engagement disk 616, which results in rotation of the intermediary force bridging interface force communication disk, which results in rotation of the endoscope-side quite release interface mating engagement disk 516, shaft 514, and pulley 512, which results in linear motion of or linear force applied to the endoscope-side tendon 334 (e.g., one side of the endoscope-side tendon 334 versus the other side of the endoscope-side tendon 334, with respect to the pulley's circumference, depending upon the direction of rotation of the endoscope-side quick release interface pulley 512). Such linear motion or linear force is communicated along the endoscope-side tendon 334 to the robot arm 400 to which the endoscope-side tendon 334 is coupled, thereby enabling selective/selectable manipulation of the robot arm 400 and its effector 405 in response to this linear motion or force.

As indicated above, quick release interfaces 500, 600, 630 are configured for mating snap-fit engagement with each other, such that they can be selectively attached to and detached from each other. Such mating snap-fit engagement can occur by way of corresponding or counterpart structural features or engagement catch elements, such as protrusions, recesses, catch elements, etc . . . , in portions of the housing 512, 612, 642 of each of the actuator-side quick release interface 600, the intermediary force bridging interface 630, and the endoscope-side quick release interface 500, in a manner readily understood by one of ordinary skill in the relevant art. FIGS. 11A-11E illustrate representative snap-fit/engagement catch elements carried by quick release interfaces 500, 600, 630 in accordance with an embodiment of the present disclosure. In various embodiments, mating snap-fit engagement elements facilitate or enable one or more physical couplings between quick release interfaces 500, 600, 630 that are at least fluid (e.g., liquid and/or air) resistant, thereby facilitating or enabling environmental segregation or isolation between actuator-side and endoscope-side elements of the system 10. In some embodiments, one or more quick release interfaces 500, 600, 630 can include sealing elements such as gaskets or o-rings to facilitate or enable an airtight seal.

An endoscope-side quick release interface 500 and/or an actuator-side quick release interface 600 can convert rotational motion into linear motion in a variety of manners. For instance, FIG. 11H is a schematic illustration of a rotational-to-linear motion conversion assembly 650 in accordance with an embodiment of the present disclosure. In an embodiment, tendons 334 can be wrapped around a disk shaft 652 such that clockwise rotation of the disk shaft 652 tightens a first tendon 334 and releases or let's out a second tendon 334, and counterclockwise rotation of the disk shaft 652 let's out the first tendon 334 and tightens the second tendon 334. By wrapping a tendon 334 around the disk shaft 650 prior to the tendon's anchor point on the disk shaft 652, the capstan effect is utilized such that friction reduces tendon tension seen at the anchor point, thereby reducing a likelihood of failure. Winding drums 654 can be tightened against each other prior to being secured to the disk shaft 652 to facilitate proper tendon tension.

An endoscope-side quick release interface 500, an actuator-side quick release interface 600, and/or an actuation controller 700 can alternatively communicate or transmit mechanical forces to a tendon 334 in a different manner. For instance, FIG. 11I is a schematic illustration of a gimbal plate mechanical force transfer assembly or structure 660 in accordance with an embodiment of the present disclosure. In an embodiment, a gimbal plate 662 coupled to a pivot mechanism 664 are configured for facilitating or enabling gimbal plate pivoting in Cartesian axes that are parallel to a plane such as a quick release interface plane, such that pivotal motion of the gimbal plate 662 can be converted into linear motion of paired tensile tendons 334 or tendon sections/segments. Such a gimbal plate 662 can be manipulated or pressed upon by a variety of mechanisms, such as a counterpart or matching tendon-driven gimbal plate on an opposite side of the quick release interface 500, 600. Thus, in an embodiment the movement or tilting of an actuator-side gimbal plate 662 at a given angle relative to an actuator-side pivot mechanism 664 in response to the translation of actuator-side tendons 334 coupled to the actuator-side gimbal plate 662 can result in corollary or counterpart proportional movement or tilting of an endoscope-side gimbal plate 662 relative to an endoscope-side pivot mechanism 662, and corresponding displacement of endoscope-side tendons 334 coupled to the endoscope-side gimbal plate 662, in a manner correlated with the translation of the actuator-side tendons 334. The actuator-side gimbal plate 662 can have an outer face that is mechanically coupled to or in contact with a counterpart or corresponding outer face of the endoscope-side gimbal plate 662. In certain embodiments, a gimbal plate structure 660 such as that shown in FIG. 11I can additionally or alternatively be a type of joint primitive that can be carried by a robot arm 400.

Aspects of a Representative Actuation Controller

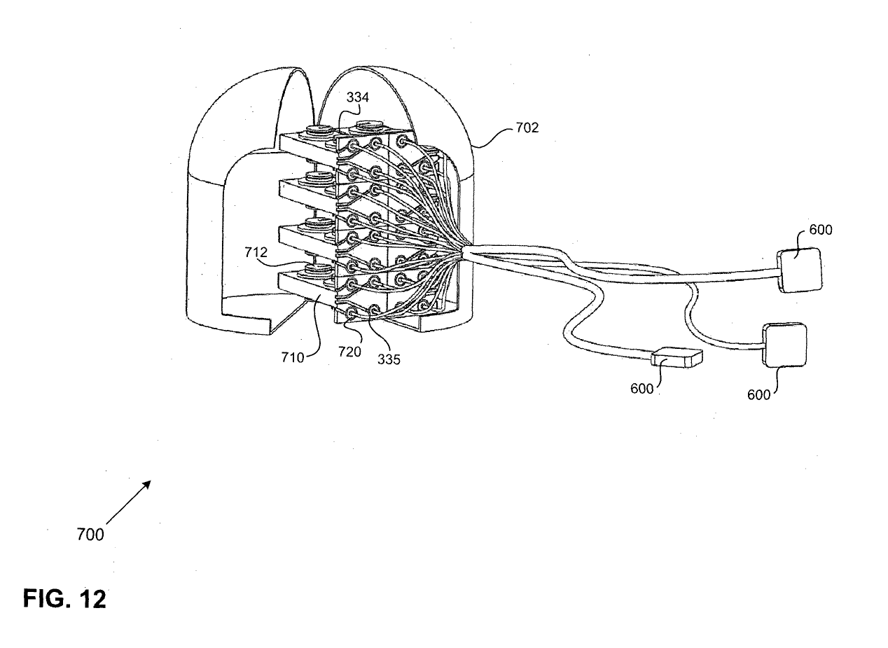

FIG. 12 is a schematic illustration of an actuation controller 700 in accordance with an embodiment of the present disclosure. In an embodiment, the actuation controller 700 includes a housing 702 that carries a set of motor/sensor assemblies 710. Each motor/sensor assembly 710 carries two motors configured for driving tensile tendon pairs or paired tendon sections/segments. In various embodiments motor can include a drum connector 712 to which a tendon 334 can be coupled, and away from which the tendon 334 extends to and through a force sensing load cell 720 as the tendon 334, within its sheath 335, further extends toward and to a corresponding actuator-side quick release interface 600.

Aspects of Representative Implementations

In representative non-limiting implementations directed to an endoscopy apparatus for a robotic master-slave surgical system, the primary endoscope probe 100 can have a length of 1.0-2.0 m, and an outer or barrel diameter of 18.0-20.0 mm. The primary endoscope probe's tool channels 130 can have a diameter of between 5.0-8.0 mm (e.g., 5.5-7.5 mm), and can (a) be separated from each other by a very small distance, or (b) touch each other in order to optimally utilize the limited internal space/volume provided by the primary endoscope probe 100. A suction channel 180 can have a diameter of 2.0-5.0 mm. The primary endoscope probe 100 can be made from one or more types of medical grade materials. For instance, the primary endoscope probe 100 can include medical grade stainless steel, which can be surrounded by or coated with one or more types of polymer materials, such as Fluorinated ethylene propylene (FEP), Polytetraflyuroethylene (PTFE), or Polyurethane (PU), to enhance lubricity and provide electrical isolation with respect to high-voltage electrosurgical instruments or elements that may be carried within the primary endoscope probe 100.

The secondary endoscope probe 200 can have a length of 150.0-250.0 cm, and an outer or barrel diameter of 3.5-8.0 mm. The primary endoscope probe's secondary endoscope probe channel 140 is thus configured to have an inner diameter that is slightly or very slightly larger (e.g., by 0.1-0.5 mm) than the outer diameter of the secondary endoscope probe 200, such that the secondary endoscope probe 200 can smoothly surge and possibly rotate/roll within the secondary endoscope probe channel 140. When the secondary endoscope probe 200 includes a set of controllable regions 230 within or spanning a distal section of the secondary endoscope probe 200, the overall length of this distal section can be 2.0-8.0 cm, and the length of a given controllable region 230 can be 0.5-2.5 cm. The secondary endoscope probe 200 can be configured for 4.0-9.0 cm of surge displacement; 1.0-4.0 cm of heave displacement; and up to 2.0 cm of sway displacement. The secondary endoscope probe 200 can be made from one or more types of medical grade materials, for instance, materials analogous to those described above with regard to the primary endoscope probe 100. In multiple implementations, the secondary endoscope probe 200 is based upon, essentially is, or is a conventional/ commercially available imaging endoscope.

In implementations in which the primary endoscope probe 100 includes a taper member/ ramp structure 144/150, the taper member/ramp structure length can be 2-14 mm; the taper member/ramp structure height can be 1.0-8.0 mm; and the articulation angle θ_{A} provided by the taper member/ramp structure is 30.0 degrees or less. A movable ramp structure 150 can be configured for displacement across a distance of 2.5-10.0 mm. A taper member/ramp structure 144/150 can be made using one or more types of medical grade materials, for instance, a material identical or analogous to that from which the primary endoscope probe 100 is made.

In implementations that include a secondary probe member 270, the secondary probe member 270 can have a length of 5.0-20.0 mm, and a width of 5.0 mm or more (e.g., 5.0-8.0 mm, corresponding to the width of a secondary endoscope probe 200; or up to 18.0-20.0 mm corresponding to the outer diameter of the primary endoscope probe 100, depending upon embodiment details). The secondary probe member 270 can be configured for heave, sway, and/or other displacement relative to the primary endoscope probe's central axis in a manner identical or analogous to that for a secondary endoscope probe 200.

A disposable actuation assembly 300 can have a length of 1.2-2.0 m. A robot arm 400 can have an outer diameter of 5.0-7.0 mm, which is generally 0.1-0.5 mm less than the inner diameter of tool channel 130 that carries the robot arm 400. A robot arm 400 can be made using one or more types of medical grade materials, such as medical grade stainless steel. Outer surfaces of the robot arm 400 can include or be coated with one or more types of polymer materials, such as FEP, PTFE, PU, and/or another material, to enhance lubricity and for purpose of electrical isolation. Joint primitives 410, 440, 450 can have lengths of 3.0-15.0 mm, and outer diameters of 5.5-7.0 mm, and can be made using one or more types of materials in a manner analogous to that for a robot arm 400. An end effector such as a grasper or gripper 405 can have a length of 5.0-25.0 mm; a width and/or thickness of 2.0-7.0 mm; a maximum opening angle of 10-200 degrees depending upon application (e.g., a needle grasper need only open enough to grasp a needle; whereas a tissue retractor may open 180 degrees); and a maximum tip-to-tip opening distance of 6.0-50.0 mm depending upon gripper length and maximum opening angle.

With respect to a quick release assembly, each of the endoscope-side quick release interface 500, the actuator-side quick release interface 600, and the intermediary interface 630 can have a length of 8.0-16.0 cm, a width of 4.0-8.0 cm, and a height of 3.0-6.0 cm. With respect to tendon-sheath elements, endoscope-side tendon-sheath elements can have a length of 1.2-1.8 m, and actuator-side tendon-sheath elements can have a length of 0.5-2.0 m.

Aspects of particular embodiments of the present disclosure address at least one aspect, problem, limitation, and/or disadvantage associated with exiting endoscopy systems and methods. While features, aspects, and/or advantages associated with certain embodiments have been described in the disclosure, other embodiments may also exhibit such features, aspects, and/or advantages, and not all embodiments need necessarily exhibit such features, aspects, and/or advantages to fall within the scope of the disclosure. It will be appreciated by a person of ordinary skill in the art that several of the above-disclosed systems, components, processes, or alternatives thereof, may be desirably combined into other different systems, components, processes, and/or applications. In addition, various modifications, alterations, and/or improvements may be made to various embodiments that are disclosed by a person of ordinary skill in the art within the scope and spirit of the present disclosure. For instance, in some embodiments, one or more portions of a quick release assembly (e.g., an actuator-side quick release interface 600 or an intermediate quick release interface 630) can carry a set of sensors (e.g., a force sensing load cell corresponding to each tendon) configured to detect forces applied to tendons and/or tendon elongation. Thus, the set of sensors can be disposed remote from the end effector(s), the robot arm(s), and the primary endoscope probe; and furthermore such sensors can be disposed separate or apart from the actuation controller 700. Such sensors can facilitate the provision of force feedback to the master console 1000, for instance, in one or more manners analogous to that described in PCT Publication No. WO 2010/138083.

1. An endoscopy apparatus comprising:
a primary endoscope probe comprising an elongate flexible body having a length, a central axis, a proximal end, a distal end, and plurality of channels therewithin extending away from the proximal end and toward the distal end of the primary endoscope probe, the plurality of channels including:
at least one tool channel configured for receiving an endoscopy tool, each tool channel having a proximal opening and a distal opening; and
a secondary endoscope probe channel configured for carrying a secondary endoscope probe, the secondary endoscope probe channel having a central axis, a proximal opening, and a distal opening,
wherein the distal opening of the secondary endoscope probe channel is proximally offset away from the distal end -of the primary endoscope probe.

2. The endoscopy apparatus of claim 1, wherein the distal opening of the secondary probe channel is proximally offset away from the distal end of the primary endoscope probe by up to 15% of the length of the primary endoscope probe.

3. The endoscopy apparatus of claim 2, wherein the distal opening of the secondary probe channel is proximally offset away from the distal end of the primary endoscope probe by up to 10% of the length of the primary endoscope probe.

4. The endoscopy apparatus of claim 1, further comprising:
an actuation assembly disposed within a tool channel of the at least one tool channel, the actuation assembly including an end effector and a set of actuation elements configured for controlling the end effector, the actuation assembly translatable along the primary endoscope probe central axis such that the end effector is disposable within a target environment beyond the distal end of the primary endoscope probe; and
a secondary endoscope probe carried within the secondary endoscope probe channel, the secondary endoscope probe having a distal end displaceable beyond the distal opening of the secondary endoscope probe channel,
wherein the secondary endoscope probe comprises an imaging endoscope configured for capturing images of the end effector within the target environment beyond the distal end of the primary endoscope probe, and
wherein the imaging endoscope includes at least one of:
at least one controllable region configured for enabling controllable displacement of the imaging endoscope toward or away from the primary endoscope probe central axis; and
an image capture module having a field of view disposed toward the central axis of the primary endoscope probe.

5. The endoscopy apparatus of claim 4, wherein the imaging endoscope is configured for capturing anterograde and retrograde views of end effector operation within the target environment.

6. The endoscopy apparatus of claim 4, wherein the at least one controllable region is configured for enabling heave displacement of the imaging endoscope relative to the primary endoscope probe central axis.

7. The endoscopy apparatus of claim 6, wherein the at least one controllable region is further configured for enabling sway displacement of the imaging endoscope relative to the primary endoscope probe central axis.

8. The endoscopy apparatus of claim 4, wherein the imaging endoscope includes a plurality of distinct controllable regions.

9. The endoscopy apparatus of claim 8, wherein the imaging endoscope comprises an S-bend endoscope.

10. The endoscopy apparatus of claim 4, wherein the imaging endoscope is configured for controllable rotation about a central axis thereof.

11. The endoscopy apparatus of claim 4, further comprising a ramp structure positioned proximate to the distal end of the primary endoscope probe and configured for receiving the imaging endoscope and guiding a central axis of the imaging endoscope toward or away from the central axis of the primary endoscope probe to thereby facilitate heave displacement of the imaging endoscope relative to the central axis of the primary endoscope probe.

12. The endoscopy apparatus of claim 11, wherein the ramp structure is controllably displaceable in a direction parallel to the central axis of the primary endoscope probe.

13. The endoscopy apparatus of claim 4, wherein the image capture module field of view is disposed toward the central axis of the primary endoscope probe by way of one of:
a beveled face carrying a lens element and positioned at a non-normal angle relative to the central axis of the secondary endoscope probe; and
a rotatable housing carrying the lens element, the rotatable housing controllably displaceable about an axis of rotation transverse to the central axis of the primary endoscope probe.

14. The endoscopy apparatus of claim 13, wherein the distal end of the primary endoscope probe is configured for mating engagement with the rotatable housing, and wherein the rotatable housing is displaceable beyond the distal end of the primary endoscope probe.

15. An endoscopy apparatus comprising:
a primary endoscope probe comprising an elongate flexible body having a central axis, a proximal end, a distal end, and plurality of channels therewithin extending away from the proximal end and toward the distal end of the primary endoscope probe, the plurality of channels including:
at least one tool channel, each tool channel having a proximal opening and a distal opening; and
a secondary endoscope probe channel configured for carrying a secondary endoscope probe, the secondary endoscope probe channel having a central axis, a proximal opening, and a distal opening; and
a ramp structure positioned proximate to the distal end of the primary endoscope probe and configured for receiving the secondary endoscope probe and guiding the central axis of the central axis of the secondary endoscope probe toward or away from the central axis of the primary endoscope probe to thereby facilitate heave displacement of the secondary endoscope probe relative to the central axis of the primary endoscope probe.

16. (canceled)

17. An imaging endoscope comprising:
a flexible body having a length, a central axis along its length, a proximal end, and a distal end; and
an image capture module disposed at the distal end of the flexible body and having a field of view that is controllably positionable toward and away from the central axis of the flexible body by way of a rotatable housing having an axis of rotation transverse to the central axis of the flexible body.

18.-21. (canceled)

22. The endoscopy apparatus of claim 4, wherein positioning of the primary endoscope probe and positioning of the secondary endoscope probe is controllable by an interface coupled to the proximal end of the primary endoscope probe, and wherein positioning of the robot arm is controllable by a master controller disposed remote from the primary endoscope probe and the interface coupled to the proximal end of the primary endoscope probe.

23.-35. (canceled)
